(19) United States
(12) Patent Application Publication
(10) Pub. No.: US 2014/0296265 A1
(43) Pub. Date: Oct. 2, 2014

(54) Method of Treating Lymphoma Using Pyridopyrimidinone Inhibitors of PI3K/mTOR

(71) Applicants: Arthur DeCillis, Madison, CT (US); Joanne Lager, Hollis, NH (US); Tal Zaks, Newton, MA (US)

(72) Inventors: Arthur DeCillis, Madison, CT (US); Joanne Lager, Hollis, NH (US); Tal Zaks, Newton, MA (US)

(73) Assignees: Sanofi, Paris (FR); Exelixis, Inc., South San Francisco, CA (US)

(21) Appl. No.: 14/114,721

(22) PCT Filed: Apr. 27, 2012
(86) PCT No.: PCT/US2012/035442

§ 371 Date: Jun. 16, 2014

Related U.S. Application Data
(60) Provisional application No. 61/480,991, filed on Apr. 29, 2011.
(60) Provisional application No. 61/493,998, filed on Jun. 7, 2011.
(60) Provisional application No. 61/566,066, filed on Dec. 2, 2011.

(51) Int. Cl. C07D 471/04

(52) U.S. Cl. CPC C07D 471/04
USPC 514/264.11; 544/279

(57) ABSTRACT

The invention provides a method for treating cancers including hematologic malignancies comprising administering a compound of formula I:

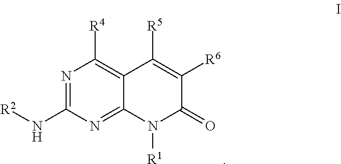

### CROSS-REFERENCE TO RELATED APPLICATIONS

This application claims the benefit of priority of U.S. Provisional Application No. 61/480,991, filed Apr. 29, 2011, U.S. Provisional Application No. 61/493,998, filed Jun. 7, 2011, and U.S. Provisional Application No. 61/566,066, filed Dec. 2, 2011, all of which are incorporated herein by reference.

### BACKGROUND

Lymphoproliferative malignancies, including lymphomas and lymphocytic leukemia, are common malignancies with an incidence of approximately 93,000 new cases a year in the United States.

Treatment modalities being developed to treat these malignancies are being met with varying levels of success. For example, among the more than 30 subtypes of non-Hodgkin lymphoma (NHL), mantle cell lymphoma (MCL) accounts for 3 percent to 10 percent of cases. MCL can be treated at diagnosis or recurrence with various chemotherapeutic regimens. Although the prognosis is improving given these advances in treatment, the median overall survival remains 4.8 years.

Follicular lymphoma (FL) is a common indolent B-cell NHL that constitutes approximately 20 percent of all newly diagnosed lymphoma cases and approximately 70 percent of all indolent NHL. Like many lymphomas, it is increasing in incidence, with over 24,000 new cases diagnosed each year. While there is an increasing number of available treatment modalities for FL, including radioimmunotherapy alone or in combination with chemotherapy, as well as bone marrow transplantation, many FL patients develop treatment-refractory disease or relapse due to molecular escape mechanisms.

B-cell chronic lymphocytic leukemia (CLL) is the most common type of adult leukemia in the United States, with approximately 15,000 new cases each year. According to the World Health Organization (WHO) classification, CLL is identical (i.e., one disease at different stages) to the mature peripheral B-cell neoplasm small lymphocytic lymphoma (SLL). In spite of various treatment options, CLL/advanced SLL is a progressive disease and once symptomatic, patients have a relatively short overall survival, ranging from 18 months to 6 years, with a 22.5 percent 10-year survival expectation.

Diffuse Large B-cell Lymphoma (DLBCL) is the most common subtype of malignant lymphoma and constitutes approximately 40% of all cases. Despite treatment improvements in the past years, approximately one third of patients with advanced-stage DLBCL will be refractory to therapy or will relapse, the vast majority of whom will die of their disease.

As a result, there is an ongoing need for clinically effective agents for treating lymphoproliferative malignancies, including lymphomas and lymphocytic leukemia, and especially lymphoproliferative malignancies that are relapsed or refractory lymphomas or lymphocytic leukemia. More particularly, there is an ongoing need for clinically effective agents for treating relapsed or refractory NHL, MCL, FL, CLL/SLL, and DLBCL.

### SUMMARY

Accordingly, methods are provided for treating lymphoproliferative malignancies, particularly, relapsed or refractory MCL, FL, CLL/SLL, and DLBCL comprising administering to a patient in need thereof a therapeutically effective amount of a compound of formula I:

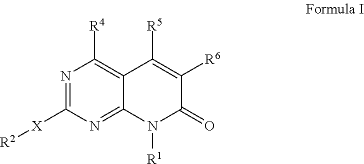

or a pharmaceutically acceptable salt, thereof; or a pharmaceutical composition comprising a therapeutically effective amount of a compound of formula I;
where the compound of formula I is that wherein:
R¹ is hydrogen, optionally substituted alkyl, optionally substituted cycloalkyl, optionally substituted cycloalkylalkyl, optionally substituted aryl, optionally substituted arylalkyl, optionally substituted heterocycloalkyl, optionally substituted heterocycloalkylalkyl, optionally substituted heteroaryl, or optionally substituted heteroarylalkyl;
R² is hydrogen or alkyl where the alkyl is optionally substituted with 1, 2, 3, 4, or 5 R⁸ groups;
X is —NR³—;
R³ is hydrogen;
R⁴ is optionally substituted alkyl;
R⁵ is hydrogen; and
R⁶ is phenyl, acyl, or heteroaryl wherein the phenyl and heteroaryl are optionally substituted with 1, 2, 3, 4, or 5 R⁹ groups;
each R⁸, when present, is independently hydroxy, halo, alkoxy, haloalkoxy, amino, alkylamino, dialkylaminoalkyl, or alkoxyalkylamino; and
each R⁹, when present, is independently halo, alkyl, haloalkyl, alkoxy, haloalkoxy, cyano, amino, alkylamino, dialkylamino, alkoxyalkyl, carboxyalkyl, alkoxycarbonyl, aminoalkyl, cycloalkyl, aryl, arylalkyl, aryloxy, heterocycloalkyl, or heteroaryl, and where the cycloalkyl, aryl, heterocycloalkyl, and heteroaryl, each either alone or as part of another group within R⁹, are independently optionally substituted with 1, 2, 3, or 4 groups selected from halo, alkyl, haloalkyl, hydroxy, alkoxy, haloalkxy, amino, alkylamino, and dialkylamino.

In another aspect, methods of treating patients having Non-Hodgkins lymphoma (NHL) mantle cell lymphoma (MCL), follicular lymphoma (FL), chronic lymphocytic leukemia/small lymphocytic lymphoma (CLL/SLL), or Diffuse Large B-cell Lymphoma (DLBCL) are provided herein, comprising administering to said patient an effective amount of a composition comprising 2-amino-8-ethyl-4-methyl-6-(1H-pyrazol-5-yl)pyrido[2,3-d]pyrimidin-7(8H)-one or a pharmaceutically acceptable salt thereof. In some embodiments, the method comprises at least one dosing cycle, wherein the dosing cycle is a period of 28 days, wherein 2-amino-8-ethyl-4-methyl-6-(1H-pyrazol-5-yl)pyrido[2,3-d]pyrimidin-7(8H)-one or a pharmaceutically acceptable salt thereof is administered at about 50 mg twice daily.

In another aspect, methods of treating human patients having mantle cell lymphoma, follicular lymphoma, chronic lymphocytic leukemia/small lymphocytic lymphoma, or Diffuse Large B-cell Lymphoma (DLBCL) are provided herein, comprising administering to said patient a clinically proven safe and effective amount of a composition comprising 2-amino-8-ethyl-4-methyl-6-(1H-pyrazol-5-yl)pyrido[2,3-d]pyrimidin-7(8H)-one or a pharmaceutically acceptable salt thereof. In some embodiments, the method comprises at least one dosing cycle, wherein the dosing cycle is a period of 28 days, wherein 2-amino-8-ethyl-4-methyl-6-(1H-pyrazol-5-yl)pyrido[2,3-d]pyrimidin-7(8H)-one or a pharmaceutically acceptable salt thereof is administered at about 50 mg twice daily.

In another aspect, methods of treating human patients having mantle cell lymphoma, follicular lymphoma, chronic lymphocytic leukemia/small lymphocytic lymphoma, or Diffuse Large B-cell Lymphoma (DLBCL) are provided herein, comprising administering to said patient an FDA approved amount of a composition comprising 2-amino-8-ethyl-4-methyl-6-(1H-pyrazol-5-yl)pyrido[2,3-d]pyrimidin-7(8H)-one or a pharmaceutically acceptable salt thereof. In some embodiments, the method comprises at least one dosing cycle, wherein the dosing cycle is a period of 28 days, wherein 2-amino-8-ethyl-4-methyl-6-(1H-pyrazol-5-yl)pyrido[2,3-d]pyrimidin-7(8H)-one or a pharmaceutically acceptable salt thereof is administered at about 50 mg twice daily.

In another aspect, a pharmaceutical composition is provided that treats human patients with mantle cell lymphoma, follicular lymphoma chronic lymphocytic leukemia/small lymphocytic lymphoma, or Diffuse Large B-cell Lymphoma (DLBCL), the composition comprising a clinically proven safe and effective amount of 2-amino-8-ethyl-4-methyl-6-(1H-pyrazol-5-yl)pyrido[2,3-d]pyrimidin-7(8H)-one or a pharmaceutically acceptable salt thereof.

### BRIEF DESCRIPTION OF THE DRAWINGS

FIG. 1 shows CT scans of a patient with MCL, pre-treatment and after two treatment cycles with Compound A.

FIG. 2 shows the mean plasma concentrations during cycle 1 on days 1 and 27 and during cycle 2 on day 22 after daily treatment.

FIG. 3 shows the PI3K/MAPK pathways inhibition and Ki67 reduction by Compound A in a mantle cell lymphoma tumor.

### DETAILED DESCRIPTION

### Abbreviations and Definitions

The following abbreviations and terms have the indicated meanings throughout:

| Abbreviation | Meaning |
|---|---|
| Ac | Acetyl |
| br | Broad |
| ° C. | Degrees Celsius |
| c- | Cyclo |
| CBZ | CarboBenZoxy = benzyloxycarbonyl |
| d | Doublet |
| dd | Doublet of doublet |
| dt | Doublet of triplet |
| DCM | Dichloromethane |
| DMA | Dimethylacetamide |
| DME | 1,2-dimethoxyethane |
| DMF | N,N-dimethylformamide |
| DMSO | Dimethyl sulfoxide |
| dppf | 1,1′-bis(diphenylphosphano)ferrocene |
| EI | Electron Impact ionization |
| g | Gram(s) |
| h or hr | Hour(s) |
| HPLC | High pressure liquid chromatography |
| L | Liter(s) |
| M | Molar or molarity |
| m | Multiplet |
| mg | Milligram(s) |
| MHz | Megahertz (frequency) |
| Min | Minute(s) |
| mL | Milliliter(s) |
| μL | Microliter(s) |
| μM | Micromole(s) or micromolar |
| mM | Millimolar |
| mmol | Millimole(s) |
| mol | Mole(s) |
| MS | Mass spectral analysis |
| N | Normal or normality |
| nM | Nanomolar |
| NMR | Nuclear magnetic resonance |
| | spectroscopy |
| q | Quartet |
| RT | Room temperature |
| s | Singlet |
| t or tr | Triplet |
| TFA | Trifluoroacetic acid |
| THF | Tetrahydrofuran |
| TLC | Thin layer chromatography |

The symbol “—” means a single bond, “═” means a double bond, “≡” means a triple bond, and “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent formula, the “ [symbol] ” or

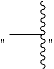

symbol will be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

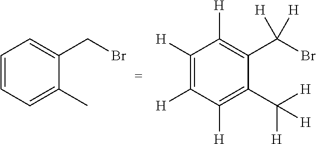

If a group “R” is depicted as “floating” on a ring system, as for example in the formula:

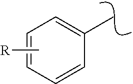

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

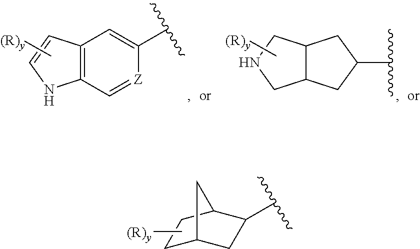

then, unless otherwise defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals=CH—) from one of the ring atoms, so long as a stable structure is formed. In the example depicted, the “R” group may reside on either the 5-membered or the 6-membered ring of the fused ring system. In the formula depicted above, when y is 2 for example, then the two “R's” may reside on any two atoms of the ring system, again assuming each replaces a depicted, implied, or expressly defined hydrogen on the ring.

When a group “R” is depicted as existing on a ring system containing saturated carbons, as for example in the formula:

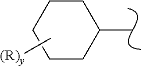

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

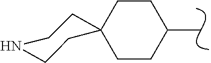

“Acyl” means a —C(O)R radical where R is optionally substituted alkyl, optionally substituted alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, trifluoromethylcarbonyl, or 2-methoxyethylcarbonyl, and the like.

“Acylamino” means a —NRR′ radical where R is hydrogen, hydroxy, alkyl, or alkoxy, and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents, “administration” and its variants are each understood to include concurrent and sequential introduction of the compound or prodrug thereof and other agents.

“Alkenyl” means a linear monovalent hydrocarbon radical of one to six carbon atoms or a branched monovalent hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, preferably one, two, or three, alkoxy groups as defined herein. Representative examples include methoxymethyl and the like.

“Alkoxyalkylamino” means an —NRR′ group where R is hydrogen, alkyl, or alkoxyalkyl, and R′ is alkoxyalkyl, as defined herein.

“Alkoxyalkylaminoalkyl” means an alkyl group substituted with at least one, specifically one or two, alkoxyalkylamino group(s), as defined herein.

“Alkoxycarbonyl” means a —C(O)R group where R is alkoxy, as defined herein.

“Alkyl” means a linear saturated monovalent hydrocarbon radical of one to six carbon atoms or a branched saturated monovalent hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylaminoalkyloxy” means an —OR group where R is alkylaminoalkyl, as defined herein.

“Alkylcarbonyl” means a —C(O)R group where R is alkyl, as defined herein.

“Alkynyl” means a linear monovalent hydrocarbon radical of one to six carbon atoms or a branched monovalent hydrocarbon radical of three to 6 carbon atoms which radical contains at least one triple bond, e.g., ethynyl, propynyl, butynyl, pentyN-2-yl and the like.

“Amino” means —NH₂.

“Aminoalkyl” means an alkyl group substituted with at least one, specifically one, two or three, amino groups.

“Aminoalkyloxy” means an —OR group where R is aminoalkyl, as defined herein.

“Aryl” means a monovalent six- to fourteen-membered, mono- or bi-carbocyclic ring, wherein the monocyclic ring is aromatic, and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Aryloxy” means an —OR group where R is aryl, as defined herein.

“Carboxyalkyl” means an alkyl group, as defined herein, substituted with at least one, specifically one or two, —C(O)OH group(s).

“Cycloalkyl” means a monocyclic or fused bicyclic, saturated or partially unsaturated (but not aromatic), monovalent hydrocarbon radical of three to ten carbon ring atoms. Fused bicyclic hydrocarbon radical includes bridged ring systems. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, or cyclohex-3-enyl, and the like.

“Cycloalkylalkyl” means an alkyl group substituted with at least one, specifically one or two, cycloalkyl group(s) as defined herein.

“Dialkylamino” means a —NRR′ radical where R and R′ are alkyl as defined herein, or an N-oxide derivative, or a protected derivative thereof, e.g., dimethylamino, diethylamino, N,N-methylpropylamino, or N,N-methylethylamino, and the like.

“Dialkylaminoalkyl” means an alkyl group substituted with one or two dialkylamino groups, as defined herein.

“Dialkylaminoalkyloxy” means an —OR group where R is dialkylaminoalkyl, as defined herein. Representative examples include 2-(N,N-diethylamino)-ethyloxy, and the like.

“Fused-polycyclic” or “fused ring system” means a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have spiro rings attached thereto via a single ring atom of the fused-polycyclic. In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Halogen” or “halo” refers to fluorine, chlorine, bromine, or iodine.

“Haloalkoxy” means an —OR′ group where R′ is haloalkyl as defined herein, e.g., trifluoromethoxy or 2,2,2-trifluoroethoxy, and the like.

“Haloalkyl” mean an alkyl group substituted with one or more halogens, specifically one to five halo atoms, e.g., trifluoromethyl, 2-chloroethyl, and 2,2-difluoroethyl, and the like.

“Heteroaryl” means a monocyclic, fused bicyclic, or fused tricyclic, monovalent radical of 5 to 14 ring atoms containing one or more, specifically one, two, three, or four ring heteroatoms independently selected from —O—, —S(O)_{N-} (n is 0, 1, or 2), —N—, —N(R^{x})—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R^{x} is hydrogen, alkyl, hydroxy, alkoxy, acyl, or alkylsulfonyl. Fused bicyclic radical includes bridged ring systems. Unless stated otherwise, the valency may be located on any atom of any ring of the heteroaryl group, valency rules permitting. When the point of valency is located on the nitrogen, R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl, and the derivatives thereof, or N-oxide or a protected derivative thereof.

“Heteroarylalkyl” means an alkyl group, as defined herein, substituted with at least one, specifically one or two heteroaryl group(s), as defined herein.

“Heteroatom” refers to O, S, N, or P.

“Heterocycloalkyl” means a saturated or partially unsaturated (but not aromatic) monovalent monocyclic group of 3 to 8 ring atoms or a saturated or partially unsaturated (but not aromatic) monovalent fused bicyclic group of 5 to 12 ring atoms in which one or more, specifically one, two, three, or four ring heteroatoms independently selected from O, S(O)ₙ (n is 0, 1, or 2), N, N(R^{y}) (where R^{y} is hydrogen, alkyl, hydroxy, alkoxy, acyl, or alkylsulfonyl), the remaining ring atoms being carbon. One or two ring carbon atoms may be replaced by a —C(O)—, —C(S)—, or —C(═NH)— group. Fused bicyclic radical includes bridged ring systems. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, and tetrahydropyranyl, and the derivatives thereof and N-oxide or a protected derivative thereof.

“Heterocycloalkylalkyl” means an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Heterocycloalkylalkyloxy means an —OR group where R is heterocycloalkylalkyl, as defined herein.

“Saturated bridged ring system” refers to a bicyclic or polycyclic ring system that is not aromatic. Such a system may contain isolated or conjugated unsaturation, but not aromatic or heteroaromatic rings in its core structure (but may have aromatic substitution thereon). For example, hexahydro-furo[3,2-b]furan, 2,3,3a,4,7,7a-hexahydro-1H-indene, 7-aza-bicyclo[2.2.1]heptane, and 1,2,3,4,4a,5,8,8a-octahydro-naphthalene are all included in the class “saturated bridged ring system.

“Spirocyclyl” or “spirocyclic ring” refers to a ring originating from a particular annular carbon of another ring. For example, as depicted below, a ring atom of a saturated bridged ring system (rings B and B′), but not a bridgehead atom, can be a shared atom between the saturated bridged ring system and a spirocyclyl (ring A) attached thereto. A spirocyclyl can be carbocyclic or heteroalicyclic.

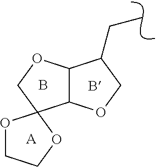

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted arylC₁₋₈ alkyl,” optional substitution may occur on both the “C₁₋₈ alkyl” portion and the “aryl” portion of the molecule, and either portion of the molecule may or may not be substituted. A list of exemplary optional substitutions is presented below in the definition of “substituted.”

“Optionally substituted alkoxy” means an —OR group where R is optionally substituted alkyl, as defined herein.

“Optionally substituted alkyl” means an alkyl radical, as defined herein, optionally substituted with one or more group(s), specifically one, two, three, four, or five groups, independently selected from alkylcarbonyl, alkenylcarbonyl, cycloalkylcarbonyl, alkylcarbonyloxy, alkenylcarbonyloxy, amino, alkylamino, dialkylamino, aminocarbonyl, alkylaminocarbonyl, dialkylaminocarbonyl, cyano, cyanoalkylaminocarbonyl, alkoxy, alkenyloxy, hydroxy, hydroxyalkoxy, halo, carboxy, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, optionally substituted alkenyl, hydroxy, alkoxy, alkenyloxy, or cyanoalkyl), alkylaminocarbonyloxy, dialkylaminocarbonyloxy, alkylaminoalkyloxy, dialkylaminoalkyloxy, alkoxycarbonyl, alkenyloxycarbonyl, alkoxycarbonylamino, alkylaminocarbonylamino, dialkylaminocarbonylamino, alkoxyalkyloxy, and —C(O)NR^{a}R^{b} (where R^{a} and R^{b} are independently hydrogen, alkyl, optionally substituted alkenyl, hydroxy, alkoxy, alkenyloxy, or cyanoalkyl).

“Optionally substituted alkenyl” means an alkyl radical, as defined herein, optionally substituted with one or more group(s), specifically one, two, three, four, or five groups, independently selected from alkylcarbonyl, alkenylcarbonyl, cycloalkylcarbonyl, alkylcarbonyloxy, alkenylcarbonyloxy, amino, alkylamino, dialkylamino, aminocarbonyl, alkylaminocarbonyl, dialkylaminocarbonyl, cyano, cyanoalkylaminocarbonyl, alkoxy, alkenyloxy, hydroxy, hydroxyalkoxy, halo, carboxy, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, optionally substituted alkenyl, hydroxy, alkoxy, alkenyloxy, or cyanoalkyl), alkylaminocarbonyloxy, dialkylaminocarbonyloxy, alkylaminoalkyloxy, dialkylaminoalkyloxy, alkoxycarbonyl, alkenyloxycarbonyl, alkoxycarbonylamino, alkylaminocarbonylamino, dialkylaminocarbonylamino, alkoxyalkyloxy, and —C(O)NR^{a}R^{b} (where R^{a} and R^{b} are independently hydrogen, alkyl, optionally substituted alkenyl, hydroxy, alkoxy, alkenyloxy, or cyanoalkyl).

“Optionally substituted amino” refers to the group —N(H)R or —N(R)R where each R is independently selected from the group: optionally substituted alkyl, optionally substituted alkoxy, optionally substituted aryl, optionally substituted heterocycloalkyl, optionally substituted heteroaryl, acyl, carboxy, alkoxycarbonyl, —S(O)₂-(optionally substituted alkyl), —S(O)₂-optionally substituted aryl), —S(O)₂-(optionally substituted heterocycloalkyl), —S(O)₂-(optionally substituted heteroaryl), and —S(O)₂-(optionally substituted heteroaryl). For example, “optionally substituted amino” includes diethylamino, methylsulfonylamino, and furanyl-oxy-sulfonamino.

“Optionally substituted aminoalkyl” means an alkyl group, as defined herein, substituted with at least one, specifically one or two, optionally substituted amino group(s), as defined herein.

“Optionally substituted aryl” means an aryl group, as defined herein, optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or aryl is pentafluorophenyl. Within the optional substituents on “aryl”, the alkyl and alkenyl, either alone or as part of another group (including, for example, the alkyl in alkoxycarbonyl), are independently optionally substituted with one, two, three, four, or five halo.

“Optionally substituted arylalkyl” means an alkyl group, as defined herein, substituted with optionally substituted aryl, as defined herein.

“Optionally substituted cycloalkyl” means a cycloalkyl group, as defined herein, substituted with one, two, or three groups independently selected from acyl, acyloxy, acylamino, optionally substituted alkyl, optionally substituted alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, and cyano. Within the above optional substituents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl.

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, and dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or aryl is pentafluorophenyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted heterocycloalkylalkyl” means an alkyl group, as defined herein, substituted with at least one, specifically one or two, optionally substituted heterocycloalkyl group(s) as defined herein.

“Lymphoproliferative malignancies” are malignant diseases of the lymphoid cells that include lymphomas and lymphocytic leukemia. Lymphoproliferative malignancies include, for instance, the more than 30 subtypes of non-Hodgkin's lymphoma (NHL) including aggressive B-cell lymphomas (e.g., Diffuse Large B-cell Lymphoma, Mantle Cell Lymphoma, and Burkitt's Lymphoma), indolent B-cell lymphomas (e.g. Follicular Lymphoma), Chronic Lymphocytic Leukemia/Small Lymphocytic Lymphoma, Mantle Cell Lymphoma (MCL), Marginal Zone Lymphomas (MZLs) (e.g., Extranodal MZL (MALT lymphoma), Nodal MZL and Splenic MZL (NCCN, 2010)); and Lymphoplasmacytic Lymphoma (also called Waldenstrom's Magroglobulinemia).

As used herein, “Compound A” means the structure

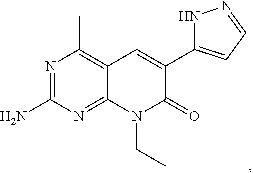 [SMILES: CCn1c(=O)c(-c2ccn[nH]2)cc2c(C)nc(N)nc21]

known by its name 2-amino-8-ethyl-4-methyl-6-(1H-pyrazol-5-yl)pyrido[2,3-d]pyrimidin-7(8H)-one. Compound A is disclosed in WO 07/044813, the entire contents of which is incorporated herein by reference.

“Pharmaceutical composition” comprises 1) a compound of formula I or a single isomer thereof where the compound is optionally as a pharmaceutically acceptable salt and additionally optionally as a hydrate and additionally optionally as a solvate thereof; and 2) a pharmaceutically acceptable carrier, excipient, or diluent as described herein.

“Yield” for each of the reactions described herein is expressed as a percentage of the theoretical yield.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

The terms “effective amount” or “pharmaceutically effective amount” or “therapeutically effective amount” refer to a sufficient amount of an agent to provide the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent, and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. For example, an “effective amount” for therapeutic uses is the amount of Compound A or a metabolite thereof, a pharmaceutically acceptable salt or solvate thereof, or a composition comprising Compound A or a metabolite thereof or a pharmaceutically acceptable salt thereof, required to provide a clinically significant decrease in relapsed or refractory MCL, FL, CLL/SLL, or DLBCL.

In some embodiments, at least one therapeutic effect is obtained. The therapeutic effect may be reduction in size of a MCL, FL, CLL/SLL, or DLBCL, reduction in metastasis, complete remission, partial remission, pathologic complete response, increase in overall response rate, or stable disease. In some embodiments, a comparable clinical benefit rate (CBR=CR+PR+SD≧6 months) is obtained by administering Compound A or a metabolite or pharmaceutically acceptable salt thereof as compared to treatment with an antitumor agent. In some embodiments, the improvement of clinical benefit rate is at least about 20 percent, 30 percent, 40 percent, 50 percent, 60 percent, 70 percent, 80 percent, or more.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts, and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

Unless otherwise indicated, “treating” or “treatment” of a disease, disorder, or syndrome, as used herein, means inhibiting the disease, disorder, or syndrome, that is, arresting its development; and relieving the disease, disorder, or syndrome, that is, causing regression of the disease, disorder, or syndrome. As is known in the art, in the context of treatment, adjustments for systemic versus localized delivery, age, body weight, general health, sex, diet, time of administration, drug interaction, and the severity of the condition may be necessary, and will be ascertainable with routine experimentation by one of ordinary skill in the art.

“Prevention” means preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

### EMBODIMENTS

The following paragraphs present a number of embodiments that can be used to practice the invention. In each instance, the embodiment includes both the recited compounds as well as individual isomers and mixtures of isomers. In addition, in each instance, the embodiment includes the pharmaceutically acceptable salts, hydrates, and/or solvates of the recited compounds and any individual isomers or mixture of isomers thereof.

In one embodiment, methods are provided for treating cancer, comprising administering to a patient an effective amount of a compound of formula I or a metabolite or a pharmaceutically acceptable salt thereof.

In one embodiment, the cancer is a lymphoproliferative malignancy.

In another embodiment, the lymphoproliferative malignancy is relapsed or refractory MCL, FL, CLL/SL, or DLBCL.

Any of the following embodiments, including the representative compounds described below, may be used to practice any of the methods disclosed herein.

### Compounds of Formula I

In one embodiment, R¹ in the compound of formula I is hydrogen, optionally substituted alkyl, optionally substituted cycloalkyl, optionally substituted cycloalkylalkyl, optionally substituted aryl, optionally substituted arylalkyl, optionally substituted heterocycloalkyl, optionally substituted heterocycloalkylalkyl, optionally substituted heteroaryl or optionally substituted heteroarylalkyl. Specifically, R¹ is hydrogen, optionally substituted alkyl, optionally substituted cycloalkyl, optionally substituted arylalkyl, or optionally substituted heterocycloalkylalkyl. More specifically, R¹ is hydrogen, alkyl, alkyl substituted with one or two hydroxy, alkyl substituted with alkoxy, cycloalkyl, arylalkyl, or heterocycloalkylalkyl. Even more specifically, R¹ is hydrogen, methyl, ethyl, propyl, isopropyl, 2-hydroxypropyl, 3-hydroxypropyl, 2-ethoxyethyl, 3-methoxypropyl, 3-ethoxypropyl, 3-isopropoxypropyl, cyclopropyl, cyclobutyl, cyclopentyl, cyclohexyl, benzyl, or 2-piperidin-1-ylethyl. Yet even more specifically, R¹ is ethyl, isopropyl, cyclopentyl, or cyclohexyl. Yet even more specifically, R¹ is ethyl.

In another embodiment, R² is hydrogen or alkyl where the alkyl is optionally substituted with 1, 2, 3, 4, or 5 R⁸ groups. Specifically, R² is hydrogen or alkyl where the alkyl is optionally substituted with one, two, or three R⁸ groups. More specifically, R² is hydrogen or alkyl where the alkyl is optionally substituted with one, two, or three R⁸ groups; and each R⁸, when present, is independently selected from amino, alkylamino, dialkylamino, and halo. Even more specifically, R² is hydrogen, methyl, ethyl, propyl, isopropyl, tert-butyl, 3-aminopropyl, 3-(N-methylamino)-propyl, 3-(N,N-dimethylamino)-propyl, 2-fluoroethyl, or 2,2,2-trifluoroethyl. Yet even more specifically, R² is hydrogen or ethyl. Yet even more preferably, R² is ethyl.

In another embodiment, R² is hydrogen.

In another embodiment, R⁴ is optionally substituted alkyl. Specifically, R⁴ is methyl or ethyl. More specifically, R⁴ is methyl.

In another embodiment, R⁶ is acyl. More specifically, R⁶ is alkylcarbonyl. Even more specifically, R⁶ is acetyl.

In another embodiment, R⁶ is phenyl optionally substituted with 1, 2, 3, 4, or 5 R⁹ groups. Specifically, R⁶ is phenyl optionally substituted with one or two R⁹ groups; and each R⁹, when present, is independently selected from aryl, halo, alkoxy, aryloxy, and haloalkyl. More specifically, R⁶ is phenyl optionally substituted with one or two R⁹ groups; and each R⁹, when present, is independently selected from phenyl, fluoro, chloro, methoxy, phenyloxy, and trifluoromethyl. Even more specifically, R⁶ is phenyl, phenyl substituted with phenyl, fluorophenyl, difluorophenyl, chlorophenyl, dichlorophenyl, phenyl substituted with chloro and fluoro, methoxyphenyl, dimethoxyphenyl, phenyloxyphenyl, or trifluoromethylphenyl. Yet even more specifically, R⁶ is phenyl, 2-phenyl-phenyl, 3-phenyl-phenyl, 4-phenyl-phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 3-chloro-4-fluoro-phenyl, 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 4-phenyloxyphenyl, 2-trifluoromethylphenyl, 3-trifluoromethylphenyl, or 4-trifluoromethylphenyl.

In another embodiment, R⁶ is heteroaryl optionally substituted with 1, 2, 3, 4, or 5 R⁹ groups.

In another embodiment, R⁶ is a 6-membered heteroaryl optionally substituted with one or two R⁹. More specifically, R⁶ is pyridinyl, pyrazinyl, pyrimidinyl, or pyridazinyl each of which is optionally substituted with one R⁹ wherein R⁹, when present, is halo. Even more specifically, R⁶ is pyridin-2-yl, pyridin-3-yl, pyridin-4-yl, 3-fluoropyridin-4-yl, pyrazin-2-yl, pyrazin-3-yl, pyrimidin-2-yl, pyrimidin-4-yl, pyrimidin-5-yl, pyridazin-3-yl, or pyridazin-4-yl, each of which is optionally substituted with one or two R⁹.

In another embodiment, R⁶ is pyrazinyl, pyrimidinyl, or pyridazinyl, each of which is optionally substituted with one R⁹ wherein R⁹, when present, is halo. Even more specifically, R⁶ is pyrazin-2-yl, pyrazin-3-yl, pyrimidin-2-yl, pyrimidin-4-yl, pyrimidin-5-yl, pyridazin-3-yl, or pyridazin-4-yl.

In another embodiment, R⁶ is a 5-membered heteroaryl optionally substituted with one or two R⁹. Specifically R⁶ is pyrazolyl, imidazolyl, thienyl, thiazolyl, oxazolyl, isoxazolyl, oxadiazolyl, furanyl, pyrrolyl, triazolyl, or tetrazolyl, each of which is optionally substituted with one R⁹ wherein R⁹, when present, is alkyl, arylalkyl, cyano, aryl, alkoxycarbonyl, or halo. More specifically, R⁶ is pyrazol-1-yl, pyrazol-3-yl, pyrazol-4-yl, pyrazol-5-yl, imidazol-1-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-1-yl, pyrrol-2-yl, pyrrol-3-yl, triazol-1-yl, triazol-4-yl, triazol-5-yl, tetrazol-1-yl, or tetrazol-5-yl; each of which is optionally substituted with one R⁹ wherein R⁹, when present, is methyl, benzyl, cyano, phenyl, N-tert-butoxycarbonyl, or chloro. Even more specifically, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with one R⁹ wherein R⁹, when present, is methyl, benzyl, cyano, phenyl, N-tert-butoxycarbonyl, or chloro.

In another embodiment, R⁶ is thienyl, pyrrolyl, furanyl, pyrazolyl, thiazolyl, isoxazolyl, imidazolyl, triazolyl, or tetrazolyl, each of which is optionally substituted with one R⁹ wherein R⁹, when present, is methyl, benzyl, cyano, phenyl, N-tert-butoxycarbonyl, or chloro. Specifically, R⁶ is thien-2-yl, thien-3-yl, pyrrol-2-yl, furan-2-yl, furan-3-yl, pyrazol-3-yl, pyrazol-4-yl, pyrazol-5-yl, thiazol-2-yl, thiazol-5-yl, isoxazol-4-yl, imidazol-5-yl, triazol-5-yl, tetrazol-5-yl, each of which is optionally substituted with one R⁹ wherein R⁹, when present, is methyl, benzyl, cyano, phenyl, N-tert-butoxycarbonyl, or chloro. More specifically, R⁶ is thien-2-yl, thien-3-yl, 5-cyano-thien-2-yl, 4-methyl-thien-2-yl, 4-methyl-thien-3-yl, 5-chloro-thien-5-yl, 5-phenyl-thien-2-yl, pyrrol-2-yl, N-tert-butoxycarbonyl-pyrrol-2-yl, N-methyl-pyrrol-2-yl, furan-2-yl, furan-3-yl, pyrazol-3-yl, pyrazol-4-yl, N-benzyl-pyrazol-4-yl, pyrazol-5-yl, thiazol-2-yl, thiazol-5-yl, isoxazol-4-yl, imidazol-5-yl, triazol-5-yl, or tetrazol-5-yl.

In another embodiment, R⁶ is thien-2-yl, thien-3-yl, pyrrol-2-yl, furan-2-yl, furan-3-yl, pyrazol-3-yl, pyrazol-4-yl, pyrazol-5-yl, thiazol-2-yl, thiazol-5-yl, isoxazol-4-yl, imidazol-5-yl, triazol-5-yl, or tetrazol-5-yl, each of which is optionally substituted with one R⁹ wherein R⁹, when present, is methyl, benzyl, cyano, phenyl, N-tert-butoxycarbonyl, or chloro.

In another embodiment, R⁶ is indolyl, benzimidazolyl, benzofuranyl, benzoxazolyl, or benzoisoxazolyl, each of which is optionally substituted with 1, 2, 3, 4, or 5 R⁹ groups. Specifically, R⁶ is indol-2-yl, indol-3-yl, indol-4-yl, indol-5-yl, indol-6-yl, indol-7-yl, benzimidazol-2-yl, benzimidazol-4-yl, benzimidazol-5-yl, benzimidazol-6-yl, benzimidazol-7-yl, benzofuran-2-yl, benzofuran-3-yl, benzofuran-4-yl, benzofuran-5-yl, benzofuran-6-yl, benzofuran-7-yl, benzoxazol-2-yl, benzoxazol-4-yl, benzoxazol-5-yl, benzoxazol-6-yl, benzoxazol-7-yl, benzoisoxazol-3-yl, benzoisoxazol-4-yl, benzoisoxazol-5-yl, benzoisoxazol-6-yl, or benzoisoxazol-7-yl; each of which is optionally substituted with 1, 2, 3, 4, or 5 R⁹ groups. More specifically, R⁶ is indol-6-yl.

In another embodiment, R¹ is hydrogen, optionally substituted alkyl, optionally substituted cycloalkyl, optionally substituted heterocycloalkylalkyl, or optionally substituted arylalkyl; X is —NH—; R² is hydrogen or alkyl where the alkyl is optionally substituted with one or two R⁸ groups; R⁴ is alkyl; R⁵ is hydrogen; R⁶ is phenyl or heteroaryl wherein the phenyl and heteroaryl are optionally substituted with one, two, or three R⁹ groups; each R⁸, when present, is independently amino, alkylamino, dialkylamino, or halo; and each R⁹, when present, is independently alkyl, arylalkyl, cyano, aryl, alkoxycarbonyl, or halo.

In another embodiment, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with 1, 2, 3, 4, or 5 R⁹ groups.

In another embodiment, R¹ is alkyl or cycloalkyl; R⁴ is methyl; and R⁶ is heteroaryl optionally substituted with one or two R⁹ groups. Specifically, each R⁹, when present, is independently alkyl, arylalkyl, cyano, aryl, alkoxycarbonyl, or halo. Specifically, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with one R⁹ wherein R⁹, when present, is methyl, benzyl, cyano, phenyl, or N-tert-butoxycarbonyl.

In another embodiment, R² is hydrogen.

In another embodiment, R² is methyl or ethyl.

In another embodiment, R¹ is alkyl or cycloalkyl; R⁴ is methyl; and R⁶ is phenyl optionally substituted with one or two R⁹ groups. Specifically each R⁹, when present, is independently halo, alkoxy, or haloalkyl.

In another embodiment, R¹ is alkyl or cycloalkyl; R⁴ is methyl; and R² is hydrogen.

In another embodiment, R¹ is alkyl or cycloalkyl; R⁴ is methyl; and R² is optionally substituted alkyl.

Representative compounds of formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Names were generated using ACD/Labs naming software 8.00 release, product version 8.08.

TABLE 1

| Example | Structure | Name |
|---|---|---|
| 1 | 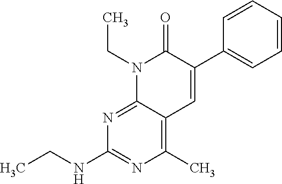 [SMILES: CCNc1nc(C)c2cc(-c3ccccc3)c(=O)n(CC)c2n1] | 8-ethyl-2-(ethylamino)-4-methyl-6- phenylpyrido[2,3-d]pyrimidin- 7(8H)-one |
| 2 | 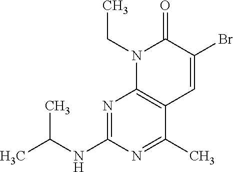 [SMILES: CCn1c(=O)c(Br)cc2c(C)nc(NC(C)C)nc21] | 6-bromo-8-ethyl-4-methyl-2-[(1- methylethyl)amino]pyrido[2,3- d]pyrimidin-7(8H)-one |
| 3 | 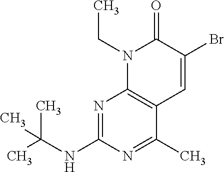 [SMILES: CCn1c(=O)c(Br)cc2c(C)nc(NC(C)(C)C)nc21] | 6-bromo-2-[(1,1- dimethylethyl)amino]-8-ethyl-4- methylpyrido[2,3-d]pyrimidin- 7(8H)-one |
| 4 | 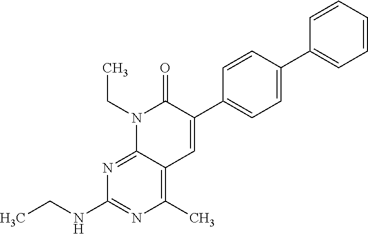 [SMILES: CCNc1nc(C)c2cc(-c3ccc(-c4ccccc4)cc3)c(=O)n(CC)c2n1] | 6-biphenyl-4-yl-8-ethyl-2- (ethylamino)-4-methylpyrido[2,3- d]pyrimidin-7(8H)-one |
| 5 | 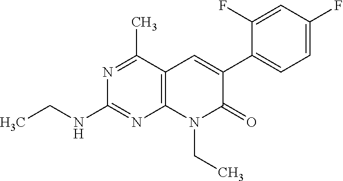 [SMILES: CCNc1nc(C)c2cc(-c3ccc(F)cc3F)c(=O)n(CC)c2n1] | 6-(2,4-difluorophenyl)-8-ethyl-2- (ethylamino)-4-methylpyrido[2,3- d]pyrimidin-7(8H)-one |
| 6 | 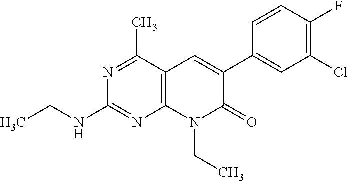 [SMILES: CCNc1nc(C)c2cc(-c3ccc(F)c(Cl)c3)c(=O)n(CC)c2n1] | 6-(3-chloro-4-fluorophenyl)-8- ethyl-2-(ethylamino)-4- methylpyrido[2,3-d]pyrimidin- 7(8H)-one |
| 7 | 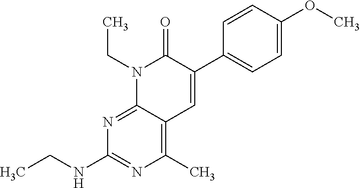 [SMILES: CCNc1nc(C)c2cc(-c3ccc(OC)cc3)c(=O)n(CC)c2n1] | 8-ethyl-2-(ethylamino)-4-methyl-6- [4-(methyloxy)phenyl]pyrido[2,3- d]pyrimidin-7(8H)-one |
| 8 | 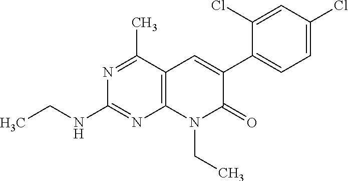 [SMILES: CCNc1nc(C)c2cc(-c3ccc(Cl)cc3Cl)c(=O)n(CC)c2n1] | 6-(2,4-dichlorophenyl)-8-ethyl-2- (ethylamino)-4-methylpyrido[2,3- d]pyrimidin-7(8H)-one |
| 9 | 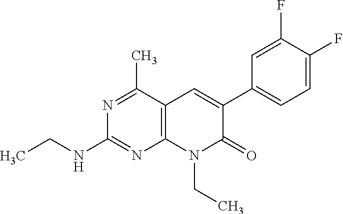 [SMILES: CCNc1nc(C)c2cc(-c3ccc(F)c(F)c3)c(=O)n(CC)c2n1] | 6-(3,4-difluorophenyl)-8-ethyl-2- (ethylamino)-4-methylpyrido[2,3- d]pyrimidin-7(8H)-one |
| 10 | 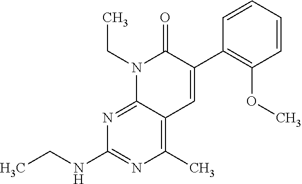 [SMILES: CCNc1nc(C)c2cc(-c3ccccc3OC)c(=O)n(CC)c2n1] | 8-ethyl-2-(ethylamino)-4-methyl-6- [2-(methyloxy)phenyl]pyrido[2,3- d]pyrimidin-7(8H)-one |
| 11 | 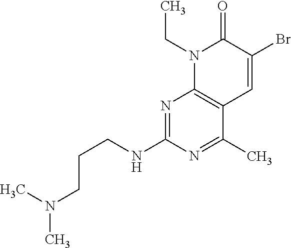 [SMILES: CCn1c(=O)c(Br)cc2c(C)nc(NCCCN(C)C)nc21] | 6-bromo-2-{[3- (dimethylamino)propyl]amino}-8- ethyl-4-methylpyrido[2,3- d]pyrimidin-7(8H)-one |
| 12 | 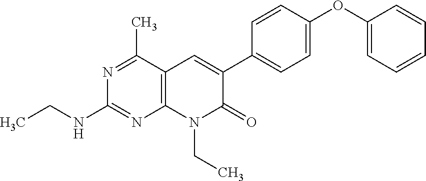 [SMILES: CCNc1nc(C)c2cc(-c3ccc(Oc4ccccc4)cc3)c(=O)n(CC)c2n1] | 8-ethyl-2-(ethylamino)-4-methyl-6- [4-(phenyloxy)phenyl]pyrido[2,3- d]pyrimidin-7(8H)-one |
| 13 | 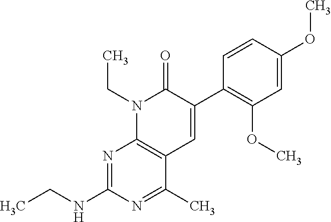 [SMILES: CCNc1nc(C)c2cc(-c3ccc(OC)cc3OC)c(=O)n(CC)c2n1] | 6-[2,4-bis(methyloxy)phenyl]-8- ethyl-2-(ethylamino)-4- methylpyrido[2,3-d]pyrimidin- 7(8H)-one |
| 14 | 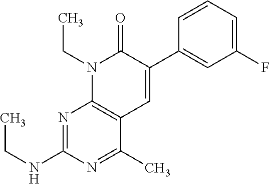 [SMILES: CCNc1nc(C)c2cc(-c3cccc(F)c3)c(=O)n(CC)c2n1] | 8-ethyl-2-(ethylamino)-6-(3- fluorophenyl)-4-methylpyrido[2,3- d]pyrimidin-7(8H)-one |
| 15 | 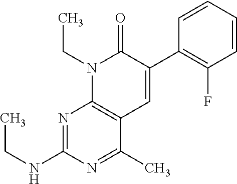 [SMILES: CCNc1nc(C)c2cc(-c3ccccc3F)c(=O)n(CC)c2n1] | 8-ethyl-2-(ethylamino)-6-(2- fluorophenyl)-4-methylpyrido[2,3- d]pyrimidin-7(8H)-one |
| 16 | 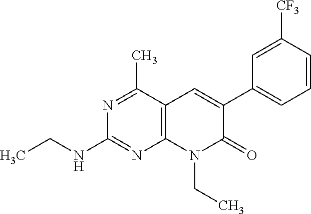 [SMILES: CCNc1nc(C)c2cc(-c3cccc(C(F)(F)F)c3)c(=O)n(CC)c2n1] | 8-ethyl-2-(ethylamino)-4-methyl-6- [3-(trifluoromethyl)phenyl]pyrido[2,3- d]pyrimidin-7(8H)-one |
| 17 | 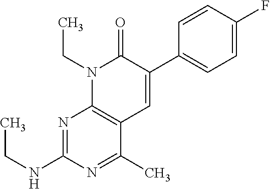 [SMILES: CCNc1nc(C)c2cc(-c3ccc(F)cc3)c(=O)n(CC)c2n1] | 8-ethyl-2-(ethylamino)-6-(4- fluorophenyl)-4-methylpyrido[2,3- d]pyrimidin-7(8H)-one |
| 18 | 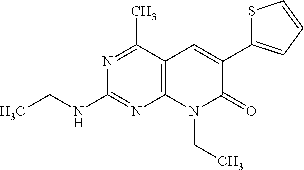 [SMILES: CCNc1nc(C)c2cc(-c3cccs3)c(=O)n(CC)c2n1] | 8-ethyl-2-(ethylamino)-4-methyl-6- (2-thienyl)pyrido[2,3-d)pyrimidin- 7(8H)-one |
| 19 | 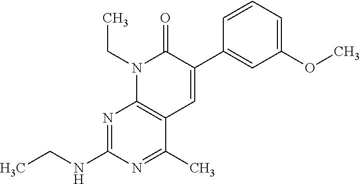 [SMILES: CCNc1nc(C)c2cc(-c3cccc(OC)c3)c(=O)n(CC)c2n1] | 8-ethyl-2-(ethylamino)-4-methyl-6- [3-(methyloxy)phenyl]pyrido[2,3- d]pyrimidin-7(8H)-one |
| 20 | 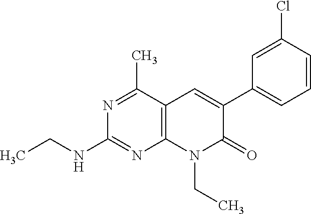 [SMILES: CCNc1nc(C)c2cc(-c3cccc(Cl)c3)c(=O)n(CC)c2n1] | 6-(3-chlorophenyl)-8-ethyl-2- (ethylamino)-4-methylpyrido[2,3- d]pyrimidin-7(8H)-one |
| 21 | 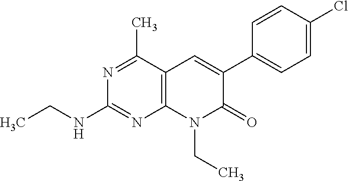 [SMILES: CCNc1nc(C)c2cc(-c3ccc(Cl)cc3)c(=O)n(CC)c2n1] | 6-(4-chlorophenyl)-8-ethyl-2- (ethylamino)-4-methylpyrido[2,3- d]pyrimidin-7(8H)-one |
| 22 | 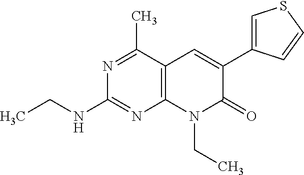 [SMILES: CCNc1nc(C)c2cc(-c3ccsc3)c(=O)n(CC)c2n1] | 8-ethyl-2-(ethylamino)-4-methyl-6- (3-thienyl)pyrido[2,3-d]pyrimidin- 7(8H)-one |
| 23 | 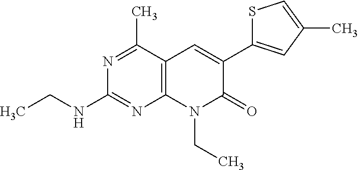 [SMILES: CCNc1nc(C)c2cc(-c3cc(C)cs3)c(=O)n(CC)c2n1] | 8-ethyl-2-(ethylamino)-4-methyl-6- (4-methyl-2-thienyl)pyrido[2,3- d]pyrimidin-7(8H)-one |
| 24 | 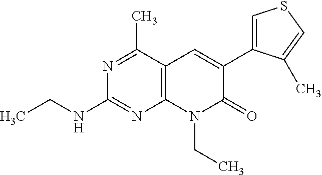 [SMILES: CCNc1nc(C)c2cc(-c3cscc3C)c(=O)n(CC)c2n1] | 8-ethyl-2-(ethylamino)-4-methyl-6- (4-methyl-3-thienyl)pyrido[2,3- d]pyrimidin-7(8H)-one |
| 25 | 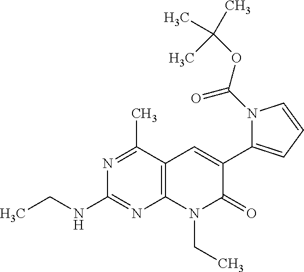 [SMILES: CCNc1nc(C)c2cc(-c3cccn3C(=O)OC(C)(C)C)c(=O)n(CC)c2n1] | 1,1-dimethylethyl 2-[8-ethyl-2- (ethylamino)-4-methyl-7-oxo-7,8- dihydropyrido[2,3-d]pyrimidin-6- yl]-1H-pyrrole-1-carboxylate |
| 26 | 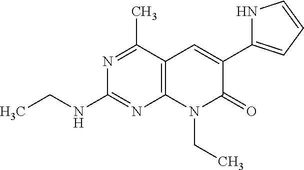 [SMILES: CCNc1nc(C)c2cc(-c3ccc[nH]3)c(=O)n(CC)c2n1] | 8-ethyl-2-(ethylamino)-4-methyl-6- (1H-pyrrol-2-yl)pyrido[2,3- d]pyrimidin-7(8H)-one |
| 27 | 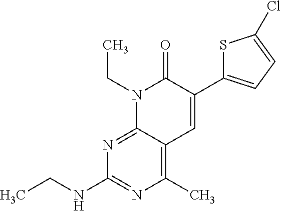 [SMILES: CCNc1nc(C)c2cc(-c3ccc(Cl)s3)c(=O)n(CC)c2n1] | 6-(5-chloro-2-thienyl)-8-ethyl-2- (ethylamino)-4-methylpyrido[2,3- d]pyrimidin-7(8H)-one |
| 28 | 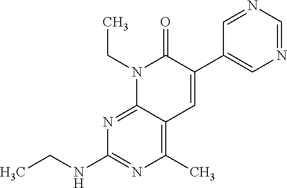 [SMILES: CCNc1nc(C)c2cc(-c3cncnc3)c(=O)n(CC)c2n1] | 8-ethyl-2-(ethylamino)-4-methyl-6- pyrimidin-5-ylpyrido[2,3- d]pyrimidin-7(8H)-one |
| 29 | 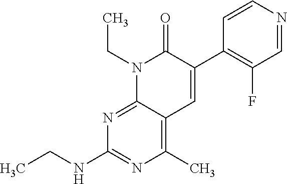 [SMILES: CCNc1nc(C)c2cc(-c3ccncc3F)c(=O)n(CC)c2n1] | 8-ethyl-2-(ethylamino)-6-(3- fluoropyridiN-4-yl)-4- methylpyrido[2,3-d]pyrimidin- 7(8H)-one |
| 30 | 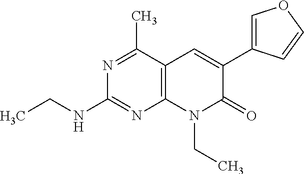 [SMILES: CCNc1nc(C)c2cc(-c3ccoc3)c(=O)n(CC)c2n1] | 8-ethyl-2-(ethylamino)-6-furan-3- yl-4-methylpyrido[2,3-d]pyrimidin- 7(8H)-one |
| 31 | 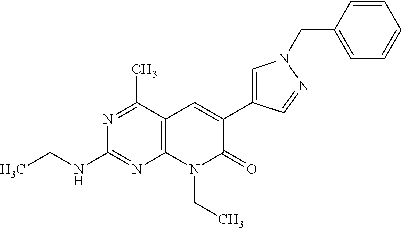 [SMILES: CCNc1nc(C)c2cc(-c3cnn(Cc4ccccc4)c3)c(=O)n(CC)c2n1] | 8-ethyl-2-(ethylamino)-4-methyl-6- [1-(phenylmethyl)-1H-pyrazol-4- yl]pyrido[2,3-d]pyrimidin-7(8H)- one |
| 32 | 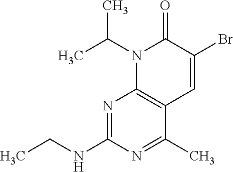 [SMILES: CCNc1nc(C)c2cc(Br)c(=O)n(C(C)C)c2n1] | 6-bromo-2-(ethylamino)-4-methyl- 8-(1-methylethyl)pyrido[12,3- d]pyrimidin-7(8H)-one |
| 33 | 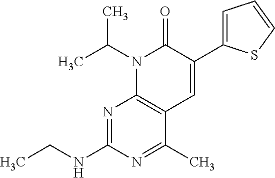 [SMILES: CCNc1nc(C)c2cc(-c3cccs3)c(=O)n(C(C)C)c2n1] | 2-(ethylamino)-4-methyl-8-(1- methylethyl)-6-(2- thienyl)pyrido[2,3-d]pyrimidin- 7(8H)-one |
| 34 | 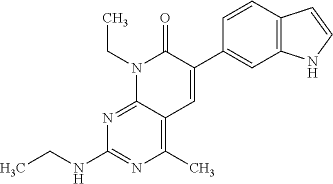 [SMILES: CCNc1nc(C)c2cc(-c3ccc4cc[nH]c4c3)c(=O)n(CC)c2n1] | 8-ethyl-2-(ethylamino)-6-(1H-indol- 6-yl)-4-methylpyrido[2,3- d]pyrimidin-7(8H)-one |
| 35 | 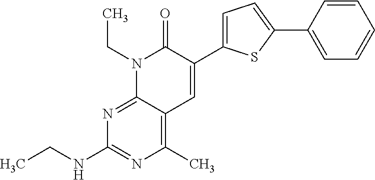 [SMILES: CCNc1nc(C)c2cc(-c3ccc(-c4ccccc4)s3)c(=O)n(CC)c2n1] | 8-ethyl-2-(ethylamino)-4-methyl-6- (5-phenyl-2-thienyl)pyrido[2,3- d]pyrimidin-7(8H)-one |
| 36 | 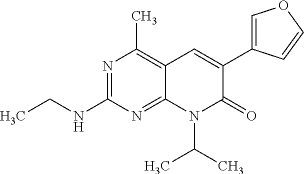 [SMILES: CCNc1nc(C)c2cc(-c3ccoc3)c(=O)n(C(C)C)c2n1] | 2-(ethylamino)-6-furan-3-yl-4- methyl-8-(1- methylethyl)pyrido[2,3- d]pyrimidin-7(8H)-one |
| 37 | 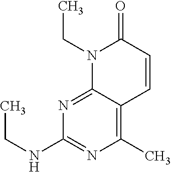 [SMILES: CCNc1nc(C)c2ccc(=O)n(CC)c2n1] | 8-ethyl-2-(ethylamino)-4- methylpyrido[2,3-d]pyrimidin- 7(8H)-one |
| 38 | 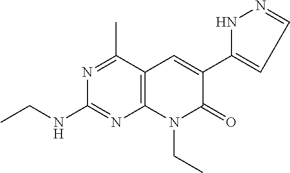 [SMILES: CCNc1nc(C)c2cc(-c3ccn[nH]3)c(=O)n(CC)c2n1] | 8-ethyl-2-(ethylamino)-4-methyl-6- (1H-pyrazol-5-yl)pyrido[2,3- d]pyrimidin-7(8H)-one |
| 39 | 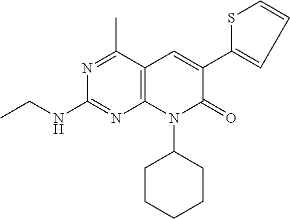 [SMILES: CCNc1nc(C)c2cc(-c3cccs3)c(=O)n(C3CCCCC3)c2n1] | 8-cyclohexyl-2-(ethylamino)-4- methyl-6-(2-thienylpyrido[2,3- d]pyrimidin-7(8H)-one |
| 40 | 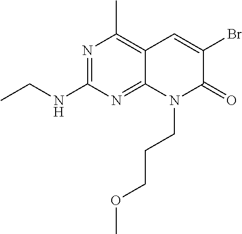 [SMILES: CCNc1nc(C)c2cc(Br)c(=O)n(CCCOC)c2n1] | 6-bromo-2-(ethylamino)-4-methyl- 8-[3-(methyloxy)propyl]pyrido[2,3- d]pyrimidin-7(8H)-one |
| 41 | 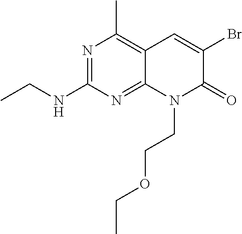 [SMILES: CCNc1nc(C)c2cc(Br)c(=O)n(CCOCC)c2n1] | 6-bromo-2-(ethylamino)-8-[2- (ethyloxy)ethyl]-4- methylpyrido[2,3-d]pyrimidin- 7(8H)-one |
| 42 | 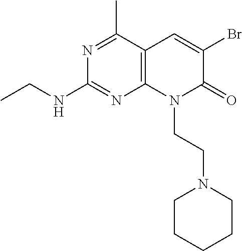 [SMILES: CCNc1nc(C)c2cc(Br)c(=O)n(CCN3CCCCC3)c2n1] | 6-bromo-2-(ethylamino)-4-methyl- 8-(2-piperidin-1-ylethyl)pyrido[2,3- d]pyrimidin-7(8H)-one |
| 43 | 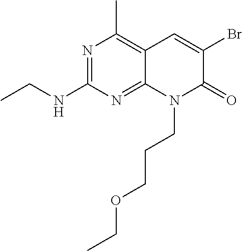 [SMILES: CCNc1nc(C)c2cc(Br)c(=O)n(CCCOCC)c2n1] | 6-bromo-2-(ethylamino)-8-[3- (ethyloxy)propyl]-4- methylpyrido[2,3-d]pyrimidin- 7(8H)-one |
| 44 | 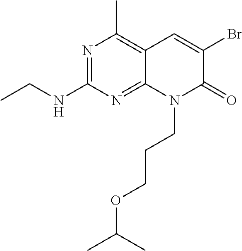 [SMILES: CCNc1nc(C)c2cc(Br)c(=O)n(CCCOC(C)C)c2n1] | 6-bromo-2-(ethylamino)-4-methyl- 8-{3-[(1- methylethyl)oxy]propyl]pyrido[2,3- d]pyrimidin-7(8H)-one |
| 45 | 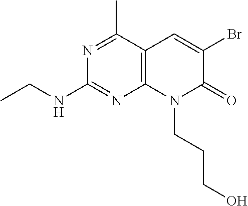 [SMILES: CCNc1nc(C)c2cc(Br)c(=O)n(CCCO)c2n1] | 6-bromo-2-(ethylamino)-8-(3- hydroxypropyl)-4- methylpyrido[2,3-d]pyrimidin- 7(8H)-one |
| 46 | 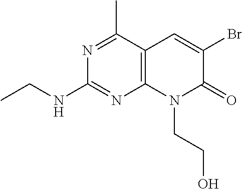 [SMILES: CCNc1nc(C)c2cc(Br)c(=O)n(CCO)c2n1] | 6-bromo-2-(ethylamino)-8-(2- hydroxyethyl)-4-methylpyrido[2,3- d]pyrimidin-7(8H)-one |
| 47 | 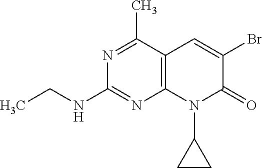 [SMILES: CCNc1nc(C)c2cc(Br)c(=O)n(C3CC3)c2n1] | 6-bromo-8-cyclopropyl-2- (ethylamino)-4-methylpyrido[2,3- d]pyrimidin-7(8H)-one |
| 48 | 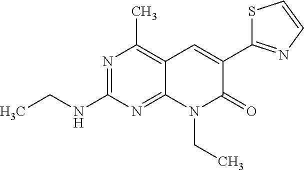 [SMILES: CCNc1nc(C)c2cc(-c3nccs3)c(=O)n(CC)c2n1] | 8-ethyl-2-(ethylamino)-4-methyl-6- (1,3-thiazol-2-yl)pyrido[2,3- d]pyrimidin-7(8H)-one |
| 49 | 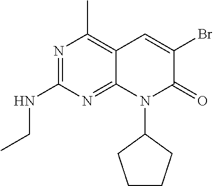 [SMILES: CCNc1nc(C)c2cc(Br)c(=O)n(C3CCCC3)c2n1] | 6-bromo-8-cyclopentyl-2- (ethylamino)-4-methylpyrido[2,3- d]pyrimidin-7(8H)-one |
| 50 | 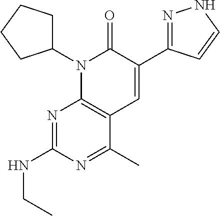 [SMILES: CCNc1nc(C)c2cc(-c3cc[nH]n3)c(=O)n(C3CCCC3)c2n1] | 8-cyclopentyl-2-(ethylamino)-4- methyl-6-(1H-pyrazol-3- yl)pyrido[2,3-d]pyrimidin-7(8H)- one |
| 51 | 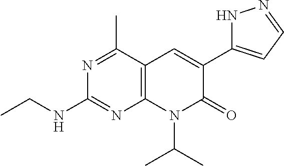 [SMILES: CCNc1nc(C)c2cc(-c3ccn[nH]3)c(=O)n(C(C)C)c2n1] | 2-(ethylamino)-4-methyl-8-(1- methylethyl)-6-(1H-pyrazol-5- yl)pyrido[2,3-d]pyrimidin-7(8H)- one |
| 52 | 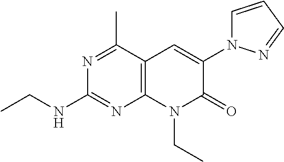 [SMILES: CCNc1nc(C)c2cc(-n3cccn3)c(=O)n(CC)c2n1] | 8-ethyl-2-(ethylamino)-4-methyl-6- (1H-pyrazol-1-yl)pyrido[2,3- d]pyrimidin-7(8H)-one |
| 53 | 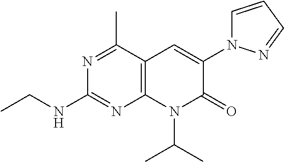 [SMILES: CCNc1nc(C)c2cc(-n3cccn3)c(=O)n(C(C)C)c2n1] | 2-(ethylamino)-4-methyl-8-(1- methylethyl)-6-(1H-pyrazol-1- yl)pyrido[2,3-d]pyrimidin-7(8H)- one |
| 54 | 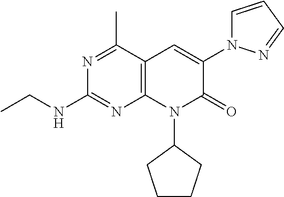 [SMILES: CCNc1nc(C)c2cc(-n3cccn3)c(=O)n(C3CCCC3)c2n1] | 8-cyclopentyl-2-(ethylamino)-4- methyl-6-(1H-pyrazol-1- yl)pyrido[2,3-d]pyrimidin-7(8H)- one |
| 55 | 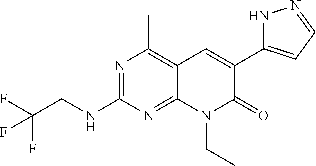 [SMILES: CCn1c(=O)c(-c2ccn[nH]2)cc2c(C)nc(NCC(F)(F)F)nc21] | 8-ethyl-4-methyl-6-(1H-pyrazol-5- yl)-2-[(2,2,2- trifluoroethyl)amino]pyrido[2,3- d]pyrimidin-7(8H)-one |
| 56 | 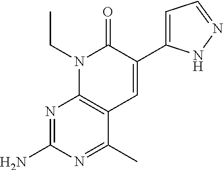 [SMILES: CCn1c(=O)c(-c2ccn[nH]2)cc2c(C)nc(N)nc21] | 2-amino-8-ethyl-4-methyl-6-(1H- pyrazol-5-yl)pyrido[2,3- d]pyrimidin-7(8H)-one |
| 57 | 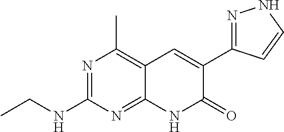 [SMILES: CCNc1nc(C)c2cc(-c3cc[nH]n3)c(=O)[nH]c2n1] | 2-(ethylamino)-4-methyl-6-(1H- pyrazol-3-yl)pyrido[2,3- d]pyrimidin-7(8H)-one |
| 58 | 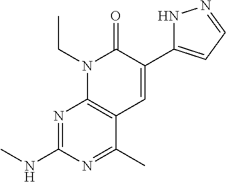 [SMILES: CCn1c(=O)c(-c2ccn[nH]2)cc2c(C)nc(NC)nc21] | 8-ethyl-4-methyl-2-(methylamino)- 6(1H-pyrazol-5-yl)pyrido[2,3- d]pyrimidin-7(8H)-one |
| 59 | 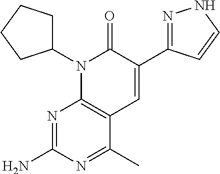 [SMILES: Cc1nc(N)nc2c1cc(-c1cc[nH]n1)c(=O)n2C1CCCC1] | 2-amino-8-cyclopentyl-4-methyl-6- (1H-pyrazol-3-yl)pyrido[2,3- d]pyrimidin-7(8H)-one |
| 60 | 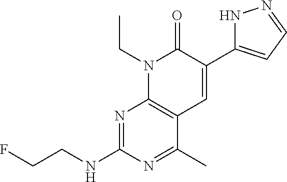 [SMILES: CCn1c(=O)c(-c2ccn[nH]2)cc2c(C)nc(NCCF)nc21] | 8-ethyl-2-[(2-fluoroethyl)amino]-4- methyl-6-(1H-pyrazol-5- yl)pyrido[2,3-d]pyrimidin-7(8H)- one |
| 61 | 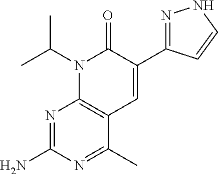 [SMILES: Cc1nc(N)nc2c1cc(-c1cc[nH]n1)c(=O)n2C(C)C] | 2-amino-4-methyl-8-(1- methylethyl)-6-(1H-pyrazol-3- yl)pyrido[2,3-d]pyrimidin-7(8H)- one |
| 62 | 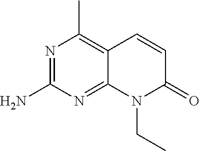 | 2-amino-8-ethyl-4- methylpyrido[2,3-d]pyrimidin- 7(8H)-one |
| 63 | 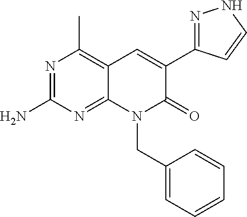 [SMILES: Cc1nc(N)nc2c1cc(-c1cc[nH]n1)c(=O)n2Cc1ccccc1] | 2-amino-4-methyl-8- (phenylmethyl)-6-(1H-pyrazol-3- yl)pyrido[2,3-d]pyrimidin-7(8H)- one |
| 64 | 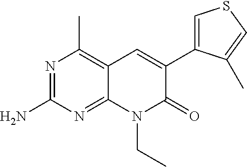 [SMILES: CCn1c(=O)c(-c2cscc2C)cc2c(C)nc(N)nc21] | 2-amino-8-ethyl-4-methyl-6-(4- methyl-3-thienyl)pyrido[2,3- d]pyrimidin-7(8H)-one |
| 65 | 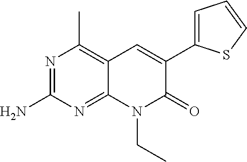 [SMILES: CCn1c(=O)c(-c2cccs2)cc2c(C)nc(N)nc21] | 2-amino-8-ethyl-4-methyl-6-(2- thienyl)pyrido[2,3-d]pyrimidin- 7(8H)-one |
| 66 | 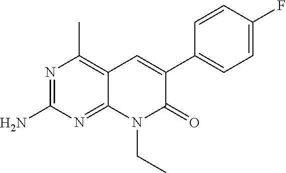 [SMILES: CCn1c(=O)c(-c2ccc(F)cc2)cc2c(C)nc(N)nc21] | 2-amino-8-ethyl-6-(4- fluorophenyl)-4-methylpyrido[2,3- d]pyrimidin-7(8H)-one |
| 67 | 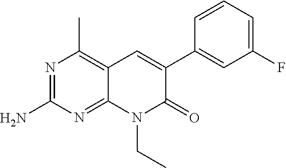 [SMILES: CCn1c(=O)c(-c2cccc(F)c2)cc2c(C)nc(N)nc21] | 2-amino-8-ethyl-6-(3- fluorophenyl)-4-methylpyrido[2,3- d]pyrimidin-7(8H)-one |
| 68 | 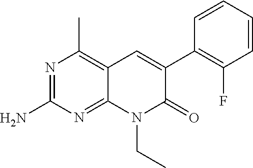 [SMILES: CCn1c(=O)c(-c2ccccc2F)cc2c(C)nc(N)nc21] | 2-amino-8-ethyl-6-(2- fluorophenyl)-4-methylpyrido[2,3- d]pyrimidin-7(8H)-one |
| 69 | 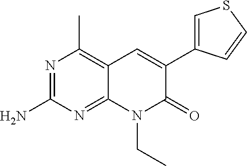 [SMILES: CCn1c(=O)c(-c2ccsc2)cc2c(C)nc(N)nc21] | 2-amino-8-ethyl-4-methyl-6-(3- thienyl)pyrido[2,3-d]pyrimidin- 7(8H)-one |
| 70 | 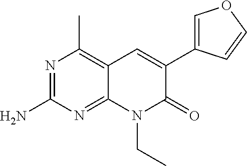 [SMILES: CCn1c(=O)c(-c2ccoc2)cc2c(C)nc(N)nc21] | 2-amino-8-ethyl-6-furan-3-yl-4- methylpyrido[2,3-d]pyrimidin- 7(8H)-one |
| 71 | 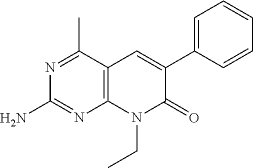 [SMILES: CCn1c(=O)c(-c2ccccc2)cc2c(C)nc(N)nc21] | 2-amino-8-ethyl-4-methyl-6- phenylpyrido[2,3-d]pyrimidin- 7(8H)-one |
| 72 | 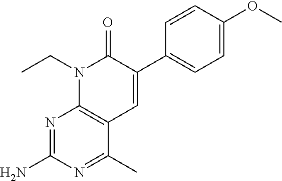 [SMILES: CCn1c(=O)c(-c2ccc(OC)cc2)cc2c(C)nc(N)nc21] | 2-amino-8-ethyl-4-methyl-6-[4- (methyloxy)phenyl]pyrido[2,3- d]pyrimidin-7(8H)-one |
| 73 | 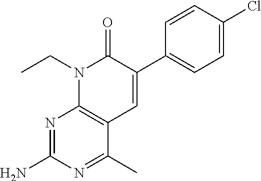 [SMILES: CCn1c(=O)c(-c2ccc(Cl)cc2)cc2c(C)nc(N)nc21] | 2-amino-6-(4-chlorophenyl)-8- ethyl-4-methylpyrido[2,3- d]pyrimidin-7(8H)-one |
| 74 | 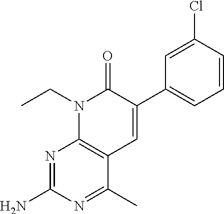 [SMILES: CCn1c(=O)c(-c2cccc(Cl)c2)cc2c(C)nc(N)nc21] | 2-amino-6-(3-chlorophenyl)-8- ethyl-4-methylpyrido[2,3- d]pyrimidin-7(8H)-one |
| 75 | 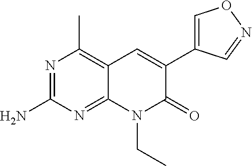 [SMILES: CCn1c(=O)c(-c2cnoc2)cc2c(C)nc(N)nc21] | 2-amino-8-ethyl-6-isoxazol-4-yl-4- methylpyrido[2,3-d]pyrimidin- 7(8H)-one |
| 76 | 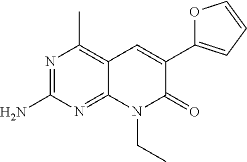 [SMILES: CCn1c(=O)c(-c2ccco2)cc2c(C)nc(N)nc21] | 2-amino-8-ethyl-6-furan-2-yl-4- methylpyrido[2,3-d]pyrimidin- 7(8H)-one |
| 77 | 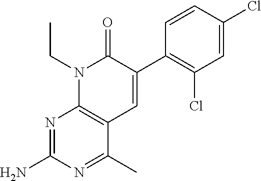 [SMILES: CCn1c(=O)c(-c2ccc(Cl)cc2Cl)cc2c(C)nc(N)nc21] | 2-amino-6-(2,4-dichlorophenyl)-8- ethyl-4-methylpyrido[2,3- d]pyrimidin-7(8H)-one |
| 78 | 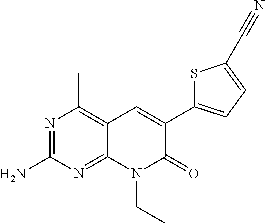 [SMILES: CCn1c(=O)c(-c2ccc(C#N)s2)cc2c(C)nc(N)nc21] | 5-(2-amino-8-ethyl-4-methyl-7-oxo- 7,8-dihydropyrido[2,3-d]pyrimidin- 6-yl)thiophene-2-carbonitrile |
| 79 | 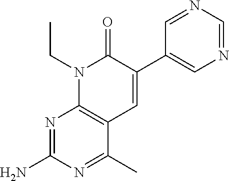 [SMILES: CCn1c(=O)c(-c2cncnc2)cc2c(C)nc(N)nc21] | 2-amino-8-ethyl-4-methyl-6- pyrimidin-5-ylpyrido[2,3- d]pyrimidin-7(8H)-one |
| 80 | 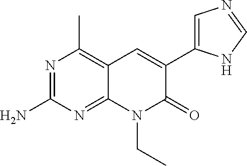 [SMILES: CCn1c(=O)c(-c2cnc[nH]2)cc2c(C)nc(N)nc21] | 2-amino-8-ethyl-6-(1H-imidazol-5- yl)-4-methylpyrido[2,3- d]pyrimidin-7(8H)-one |
| 81 | 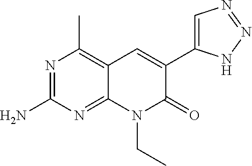 [SMILES: CCn1c(=O)c(-c2cnn[nH]2)cc2c(C)nc(N)nc21] | 2-amino-8-ethyl-4-methyl-6-(1H- 1,2,3-triazol-5-yl)pyrido[2,3- d]pyrimidin-7(8H)-one |
| 82 | 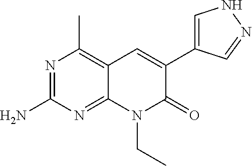 [SMILES: CCn1c(=O)c(-c2cn[nH]c2)cc2c(C)nc(N)nc21] | 2-amino-8-ethyl-4-methyl-6-(1H- pyrazol-4-yl)pyrido[2,3- d]pyrimidin-7(8H)-one |
| 83 | 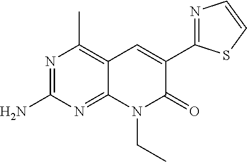 [SMILES: CCn1c(=O)c(-c2nccs2)cc2c(C)nc(N)nc21] | 2-amino-8-ethyl-4-methyl-6-(1,3- thiazol-2-yl)pyrido[2,3- d]pyrimidin-7(8H)-one |
| 84 | 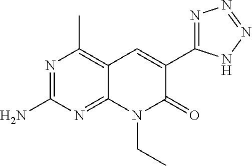 [SMILES: CCn1c(=O)c(-c2nnn[nH]2)cc2c(C)nc(N)nc21] | 2-amino-8-ethyl-4-methyl-6-(1H- tetrazol-5-yl)pyrido[2,3- d]pyrimidin-7(8H)-one |
| 85 | 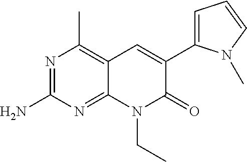 [SMILES: CCn1c(=O)c(-c2cccn2C)cc2c(C)nc(N)nc21] | 2-amino-8-ethyl-4-methyl-6-(1- methyl-1H-pyrrol-2-yl)pyrido[2,3- dipyrimidin-7(8H)-one |
| 86 | 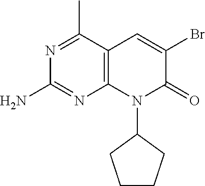 [SMILES: Cc1nc(N)nc2c1cc(Br)c(=O)n2C1CCCC1] | 2-amino-6-bromo-8-cyclopentyl-4- methylpyrido[2,3-d]pyrimidin- 7(8H)-one |
| 87 | 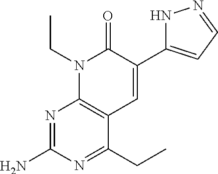 [SMILES: CCc1nc(N)nc2c1cc(-c1ccn[nH]1)c(=O)n2CC] | 2-amino-4,8-diethyl-6-(1H-pyrazol- 5-yl)pyrido[2,3-d]pyrimidin-7(8H)- one |
| 88 | 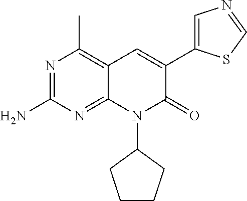 [SMILES: Cc1nc(N)nc2c1cc(-c1cncs1)c(=O)n2C1CCCC1] | 2-amino-8-cyclopentyl-4-methyl-6- (1,3-thiazol-5-yl)pyrido[2,3- d]pyrimidin-7(8H)-one |

### Compounds of Formula IA

In another embodiment, the compound of formula I is a compound of formula IA.

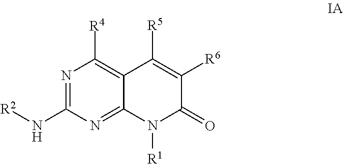

or a pharmaceutically acceptable salt thereof, wherein:

R¹ is alkyl, cycloalkyl, cycloalkylalkyl, aryl, arylalkyl, heterocycloalkyl, heterocycloalkylalkyl, heteroaryl, or heteroarylalkyl;

R² is hydrogen or alkyl;

R⁴ is alkyl;

R⁵ is hydrogen;

R⁶ is phenyl, acyl, or heteroaryl wherein the phenyl and heteroaryl are is optionally substituted with 1, 2, 3, 4, or 5 R⁹ groups; and

each R⁹, when present, is independently halo, alkyl, haloalkyl, alkoxy, haloalkoxy, cyano, amino, alkylamino, dialkylamino, alkoxyalkyl, carboxyalkyl, alkoxycarbonyl, aminoalkyl, cycloalkyl, aryl, arylalkyl, aryloxy, heterocycloalkyl, or heteroaryl and where the cycloalkyl, aryl, heterocycloalkyl, and heteroaryl, each either alone or as part of another group within R⁹, are independently optionally substituted with 1, 2, 3, or 4 groups selected from halo, alkyl, haloalkyl, hydroxy, alkoxy, haloalkoxy, amino, alkylamino, and dialkylamino.

In one embodiment, R¹ is alkyl, cycloalkyl, heterocycloalkylalkyl, or arylalkyl; X is —NH—; R² is hydrogen or alkyl; R⁴ is alkyl; R⁵ is hydrogen; R⁶ is phenyl or heteroaryl wherein the phenyl and heteroaryl are is optionally substituted with one, two, or three R⁹ groups; each R⁸, when present, is independently amino, alkylamino, dialkylamino, or halo; and each R⁸, when present, is independently alkyl, arylalkyl, cyano, aryl, or alkoxycarbonyl.

In another embodiment, R⁴ is methyl.

In another embodiment, R¹ is alkyl, cycloalkyl, or heterocycloalkyl.

In another embodiment, R¹ is alkyl.

In another embodiment, R⁶ is heteroaryl optionally substituted with 1, 2, or 3 R⁹ groups.

In another embodiment, each R⁹, when present, is independently alkyl, arylalkyl, cyano, aryl, alkoxycarbonyl, or halo.

In another embodiment, R⁶ is pyrazolyl, imidazolyl, thienyl, thiazolyl, oxazolyl, isoxazolyl, oxadiazolyl, furanyl, pyrrolyl, triazolyl, or tetrazolyl; each of which is optionally substituted with 1, 2, or 3 R⁹ groups.

In another embodiment, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with 1, 2, or 3 R⁹ groups.

In another embodiment, R⁶ is pyrazinyl, pyrimidinyl, or pyridazinyl each of which is optionally substituted with 1, 2, or 3 R⁹ groups and R⁴ is methyl.

In another embodiment, R² is hydrogen, R⁴ is methyl, R¹ is optionally substituted alkyl, cycloalkyl, or heterocycloalkyl, and R⁶ is heteroaryl optionally substituted with 1, 2, or 3 R⁹ groups.

In another embodiment, the compound of formula IA is selected from:
8-ethyl-2-(ethylamino)-4-methyl-6-phenylpyrido[2,3-d]pyrimidin-7(8H)-one;
6-biphenyl-4-yl-8-ethyl-2-(ethylamino)-4-methylpyrido[2,3-d]pyrimidin-7(8H)-one;
6-(2,4-difluorophenyl)-8-ethyl-2-(ethylamino)-4-methylpyrido[2,3-d]pyrimidin-7(8H)-one;
6-(3-chloro-4-fluorophenyl)-8-ethyl-2-(ethylamino)-4-methylpyrido[2,3-d]pyrimidin-7(8H)-one;
8-ethyl-2-(ethylamino)-4-methyl-6-[4-(methyloxy)phenyl]pyrido[2,3-d]pyrimidin-7(8H)-one;
6-(2,4-dichlorophenyl)-8-ethyl-2-(ethylamino)-4-methylpyrido[2,3-d]pyrimidin-7(8H)-one;
6-(3,4-difluorophenyl)-8-ethyl-2-(ethylamino)-4-methylpyrido[2,3-d]pyrimidin-7(8H)-one;
8-ethyl-2-(ethylamino)-4-methyl-6-[2-(methyloxy)phenyl]pyrido[2,3-d]pyrimidin-7(8H)-one;
8-ethyl-2-(ethylamino)-4-methyl-6-[4-(phenyloxy)phenyl]pyrido[2,3-d]pyrimidin-7(8H)-one;
6-[2,4-bis(methyloxy)phenyl]-8-ethyl-2-(ethylamino)-4-methylpyrido[2,3-d]pyrimidin-7(8H)-one;
8-ethyl-2-(ethylamino)-6-(3-fluorophenyl)-4-methylpyrido[2,3-d]pyrimidin-7(8H)-one;
8-ethyl-2-(ethylamino)-6-(2-fluorophenyl)-4-methylpyrido[2,3-d]pyrimidin-7(8H)-one;
8-ethyl-2-(ethylamino)-4-methyl-6-[3-(trifluoromethyl)phenyl]pyrido[2,3-d]pyrimidin-7(8H)-one;
8-ethyl-2-(ethylamino)-6-(4-fluorophenyl)-4-methylpyrido[2,3-d]pyrimidin-7(8H)-one;
8-ethyl-2-(ethylamino)-4-methyl-6-(2-thienyl)pyrido[2,3-d]pyrimidin-7(8H)-one;
8-ethyl-2-(ethylamino)-4-methyl-6-[3-(methyloxy)phenyl]pyrido[2,3-d]pyrimidin-7(8H)-one;
6-(3-chlorophenyl)-8-ethyl-2-(ethylamino)-4-methylpyrido[2,3-d]pyrimidin-7(8H)-one;
6-(4-chlorophenyl)-8-ethyl-2-(ethylamino)-4-methylpyrido[2,3-d]pyrimidin-7(8H)-one;
8-ethyl-2-(ethylamino)-4-methyl-6-(3-thienyl)pyrido[2,3-d]pyrimidin-7(8H)-one;
8-ethyl-2-(ethylamino)-4-methyl-6-(4-methyl-2-thienyl)pyrido[2,3-d]pyrimidin-7(8H)-one;
8-ethyl-2-(ethylamino)-4-methyl-6-(4-methyl-3-thienyl)pyrido[2,3-d]pyrimidin-7(8H)-one;
1,1-dimethylethyl 2-[8-ethyl-2-(ethylamino)-4-methyl-7-oxo-7,8-dihydropyrido[2,3-d]pyrimidin-6-yl]-1H-pyrrole-1-carboxylate;
8-ethyl-2-(ethylamino)-4-methyl-6-(1H-pyrrol-2-yl)pyrido[2,3-d]pyrimidin-7(8H)-one;
6-(5-chloro-2-thienyl)-8-ethyl-2-(ethylamino)-4-methylpyrido[2,3-d]pyrimidin-7(8H)-one;
8-ethyl-2-(ethylamino)-4-methyl-6-pyrimidin-5-ylpyrido[2,3-d]pyrimidin-7(8H)-one;
8-ethyl-2-(ethylamino)-6-(3-fluoropyridn-4-yl)-4-methylpyrido[2,3-d]pyrimidin-7(8H)-one;
8-ethyl-2-(ethylamino)-6-furan-3-yl-4-methylpyrido[2,3-d]pyrimidin-7(8H)-one;
8-ethyl-2-(ethylamino)-4-methyl-6-[1-(phenylmethyl)-1H-pyrazol-4-yl]pyrido[2,3-d]pyrimidin-7(8H)-one;
2-(ethylamino)-4-methyl-8-(1-methylethyl)-6-(2-thienyl)pyrido[2,3-d]pyrimidin-7(8H)-one;
8-ethyl-2-(ethylamino)-6-(1H-indol-6-yl)-4-methylpyrido[2,3-d]pyrimidin-7(8H)-one;
8-ethyl-2-(ethylamino)-4-methyl-6-(5-phenyl-2-thienyl)pyrido[2,3-d]pyrimidin-7(8H)-one;
2-(ethylamino)-6-furan-3-yl-4-methyl-8-(1-methylethyl)pyrido[2,3-d]pyrimidin-7(8H)-one;
8-ethyl-2-(ethylamino)-4-methyl-6-(1H-pyrazol-5-yl)pyrido[2,3-d]pyrimidin-7(8H)-one;
8-cyclohexyl-2-(ethylamino)-4-methyl-6-(2-thienyl)pyrido[2,3-d]pyrimidin-7(8H)-one;
8-ethyl-2-(ethylamino)-4-methyl-6-(1,3-thiazol-2-yl)pyrido[2,3-d]pyrimidin-7(8H)-one;
8-cyclopentyl-2-(ethylamino)-4-methyl-6-(1H-pyrazol-3-yl)pyrido[2,3-d]pyrimidin-7(8H)-one;
2-(ethylamino)-4-methyl-8-(1-methylethyl)-6-(1H-pyrazol-5-yl)pyrido[2,3-d]pyrimidin-7(8H)-one;
8-ethyl-2-(ethylamino)-4-methyl-6-(1H-pyrazol-1-yl)pyrido[2,3-d]pyrimidin-7(8H)-one;
2-(ethylamino)-4-methyl-8-(1-methylethyl)-6-(1H-pyrazol-1-yl)pyrido[2,3-d]pyrimidin-7(8H)-one;
8-cyclopentyl-2-(ethylamino)-4-methyl-6-(1H-pyrazol-1-yl)pyrido[2,3-d]pyrimidin-7(8H)-one;
8-ethyl-4-methyl-6-(1H-pyrazol-5-yl)-2-[(2,2,2-trifluoroethyl)amino]pyrido[2,3-d]pyrimidin-7(8H)-one;
2-amino-8-ethyl-4-methyl-6-(1H-pyrazol-5-yl)pyrido[2,3-d]pyrimidin-7(8H)-one;
2-(ethylamino)-4-methyl-6-(1H-pyrazol-3-yl)pyrido[2,3-d]pyrimidin-7(8H)-one;
8-ethyl-4-methyl-2-(methylamino)-6-(1H-pyrazol-5-yl)pyrido[2,3-d]pyrimidin-7(8H)-one;
2-amino-8-cyclopentyl-4-methyl-6-(1H-pyrazol-3-yl)pyrido[2,3-d]pyrimidin-7(8H)-one;
8-ethyl-2-[(2-fluoroethyl)amino]-4-methyl-6-(1H-pyrazol-5-yl)pyrido[2,3-d]pyrimidin-7(8H)-one;
2-amino-4-methyl-8-(1-methylethyl)-6-(1H-pyrazol-3-yl)pyrido[2,3-d]pyrimidin-7(8H)-one;
2-amino-4-methyl-8-(phenylmethyl)-6-(1H-pyrazol-3-yl)pyrido[2,3-d]pyrimidin-7(8H)-one;
2-amino-8-ethyl-4-methyl-6-(4-methyl-3-thienyl)pyrido[2,3-d]pyrimidin-7(8H)-one;
2-amino-8-ethyl-4-methyl-6-(2-thienyl)pyrido[2,3-d]pyrimidin-7(8H)-one;
2-amino-8-ethyl-6-(4-fluorophenyl)-4-methylpyrido[2,3-d]pyrimidin-7(8H)-one;
2-amino-8-ethyl-6-(3-fluorophenyl)-4-methylpyrido[2,3-d]pyrimidin-7(8H)-one;
2-amino-8-ethyl-6-(2-fluorophenyl)-4-methylpyrido[2,3-d]pyrimidin-7(8H)-one;
2-amino-8-ethyl-4-methyl-6-(3-thienyl)pyrido[2,3-d]pyrimidin-7(8H)-one;
2-amino-8-ethyl-6-furan-3-yl-4-methylpyrido[2,3-d]pyrimidin-7(8H)-one;
2-amino-8-ethyl-4-methyl-6-phenylpyrido[2,3-d]pyrimidin-7(8H)-one;
2-amino-8-ethyl-4-methyl-6-[4-(methyloxy)phenyl]pyrido[2,3-d]pyrimidin-7(8H)-one;
2-amino-6-(4-chlorophenyl)-8-ethyl-4-methylpyrido[2,3-d]pyrimidin-7(8H)-one;
2-amino-6-(3-chlorophenyl)-8-ethyl-4-methylpyrido[2,3-d]pyrimidin-7(8H)-one;
2-amino-8-ethyl-6-isoxazol-4-yl-4-methylpyrido[2,3-d]pyrimidin-7(8H)-one;
2-amino-8-ethyl-6-furan-2-yl-4-methylpyrido[2,3-d]pyrimidin-7(8H)-one;
2-amino-6-(2,4-dichlorophenyl)-8-ethyl-4-methylpyrido[2,3-d]pyrimidin-7(8H)-one;
5-(2-amino-8-ethyl-4-methyl-7-oxo-7,8-dihydropyrido[2,3-d]pyrimidin-6-yl)thiophene-2-carbonitrile;
2-amino-8-ethyl-4-methyl-6-pyrimidin-5-ylpyrido[2,3-d]pyrimidin-7(8H)-one;
2-amino-8-ethyl-6-(1H-imidazol-5-yl)-4-methylpyrido[2,3-d]pyrimidin-7(8H)-one;
2-amino-8-ethyl-4-methyl-6-(1H-1,2,3-triazol-5-yl)pyrido[2,3-d]pyrimidin-7(8H)-one;
2-amino-8-ethyl-4-methyl-6-(1H-pyrazol-4-yl)pyrido[2,3-d]pyrimidin-7(8H)-one;
2-amino-8-ethyl-4-methyl-6-(1,3-thiazol-2-yl)pyrido[2,3-d]pyrimidin-7(8H)-one;
2-amino-8-ethyl-4-methyl-6-(1H-tetrazol-5-yl)pyrido[2,3-d]pyrimidin-7(8H)-one;
2-amino-8-ethyl-4-methyl-6-(1-methyl-1H-pyrrol-2-yl)pyrido[2,3-d]pyrimidin-7(8H)-one;
2-amino-4,8-diethyl-6-(1H-pyrazol-5-yl)pyrido[2,3-d]pyrimidin-7(8H)-one; and
2-amino-8-cyclopentyl-4-methyl-6-(1,3-thiazol-5-yl)pyrido[2,3-d]pyrimidin-7(8H)-one.
In another embodiment, the compound of formula IA is selected from:
8-ethyl-2-(ethylamino)-4-methyl-6-(2-thienyl)pyrido[2,3-d]pyrimidin-7(8H)-one;
8-ethyl-2-(ethylamino)-4-methyl-6-(3-thienyl)pyrido[2,3-d]pyrimidin-7(8H)-one;
8-ethyl-2-(ethylamino)-4-methyl-6-(4-methyl-2-thienyl)pyrido[2,3-d]pyrimidin-7(8H)-one;
8-ethyl-2-(ethylamino)-4-methyl-6-(4-methyl-3-thienyl)pyrido[2,3-d]pyrimidin-7(8H)-one;
1,1-dimethylethyl 2-[8-ethyl-2-(ethylamino)-4-methyl-7-oxo-7,8-dihydropyrido[2,3-d]pyrimidin-6-yl]-1H-pyrrole-1-carboxylate
8-ethyl-2-(ethylamino)-4-methyl-6-(1H-pyrrol-2-yl)pyrido[2,3-d]pyrimidin-7(8H)-one;
6-(5-chloro-2-thienyl)-8-ethyl-2-(ethylamino)-4-methylpyrido[2,3-d]pyrimidin-7(8H)-one;
8-ethyl-2-(ethylamino)-4-methyl-6-pyrimidin-5-ylpyrido[2,3-d]pyrimidin-7(8H)-one;
8-ethyl-2-(ethylamino)-6-(3-fluoropyridn-4-yl)-4-methylpyrido[2,3-d]pyrimidin-7(8H)-one;
8-ethyl-2-(ethylamino)-6-furan-3-yl-4-methylpyrido[2,3-d]pyrimidin-7(8H)-one;
2-(ethylamino)-4-methyl-8-(1-methylethyl)-6-(2-thienyl)pyrido[2,3-d]pyrimidin-7(8H)-one;
8-ethyl-2-(ethylamino)-6-(1H-indol-6-yl)-4-methylpyrido[2,3-d]pyrimidin-7(8H)-one;
8-ethyl-2-(ethylamino)-4-methyl-6-(5-phenyl-2-thienyl)pyrido[2,3-d]pyrimidin-7(8H)-one;
2-(ethylamino)-6-furan-3-yl-4-methyl-8-(1-methylethyl)pyrido[2,3-d]pyrimidin-7(8H)-one;
8-ethyl-2-(ethylamino)-4-methyl-6-(1H-pyrazol-5-yl)pyrido[2,3-d]pyrimidin-7(8H)-one;
8-cyclohexyl-2-(ethylamino)-4-methyl-6-(2-thienyl)pyrido[2,3-d]pyrimidin-7(8H)-one;
8-ethyl-2-(ethylamino)-4-methyl-6-(1,3-thiazol-2-yl)pyrido[2,3-d]pyrimidin-7(8H)-one;
8-cyclopentyl-2-(ethylamino)-4-methyl-6-(1H-pyrazol-3-yl)pyrido[2,3-d]pyrimidin-7(8H)-one;
2-(ethylamino)-4-methyl-8-(1-methylethyl)-6-(1H-pyrazol-5-yl)pyrido[2,3-d]pyrimidin-7(8H)-one;
8-ethyl-2-(ethylamino)-4-methyl-6-(1H-pyrazol-1-yl)pyrido[2,3-d]pyrimidin-7(8H)-one;
2-(ethylamino)-4-methyl-8-(1-methylethyl)-6-(1H-pyrazol-1-yl)pyrido[2,3-d]pyrimidin-7(8H)-one;
8-cyclopentyl-2-(ethylamino)-4-methyl-6-(1H-pyrazol-1-yl)pyrido[2,3-d]pyrimidin-7(8H)-one;
8-ethyl-4-methyl-6-(1H-pyrazol-5-yl)-2-[(2,2,2-trifluoroethyl)amino]pyrido[2,3-d]pyrimidin-7(8H)-one;
2-(ethylamino)-4-methyl-6-(1H-pyrazol-3-yl)pyrido[2,3-d]pyrimidin-7(8H)-one;
8-ethyl-4-methyl-2-(methylamino)-6-(1H-pyrazol-5-yl)pyrido[2,3-d]pyrimidin-7(8H)-one; and
8-ethyl-2-[(2-fluoroethyl)amino]-4-methyl-6-(1H-pyrazol-5-yl)pyrido[2,3-d]pyrimidin-7(8H)-one.

In another embodiment, the compound of formula IA is selected form:
2-amino-8-ethyl-4-methyl-6-(1H-pyrazol-5-yl)pyrido[2,3-d]pyrimidin-7(8H)-one;
2-amino-8-cyclopentyl-4-methyl-6-(1H-pyrazol-3-yl)pyrido[2,3-d]pyrimidin-7(8H)-one;
2-amino-4-methyl-8-(1-methylethyl)-6-(1H-pyrazol-3-yl)pyrido[2,3-d]pyrimidin-7(8H)-one;
2-amino-4-methyl-8-(phenylmethyl)-6-(1H-pyrazol-3-yl)pyrido[2,3-d]pyrimidin-7(8H)-one;
2-amino-8-ethyl-4-methyl-6-(4-methyl-3-thienyl)pyrido[2,3-d]pyrimidin-7(8H)-one;
2-amino-8-ethyl-4-methyl-6-(2-thienyl)pyrido[2,3-d]pyrimidin-7(8H)-one;
2-amino-8-ethyl-4-methyl-6-(3-thienyl)pyrido[2,3-d]pyrimidin-7(8H)-one;
2-amino-8-ethyl-6-furan-3-yl-4-methylpyrido[2,3-d]pyrimidin-7(8H)-one;
2-amino-8-ethyl-4-methyl-6-phenylpyrido[2,3-d]pyrimidin-7(8H)-one;
2-amino-8-ethyl-6-isoxazol-4-yl-4-methylpyrido[2,3-d]pyrimidin-7(8H)-one;
2-amino-8-ethyl-6-furan-2-yl-4-methylpyrido[2,3-d]pyrimidin-7(8H)-one;
5-(2-amino-8-ethyl-4-methyl-7-oxo-7,8-dihydropyrido[2,3-d]pyrimidin-6-yl)thiophene-2-carbonitrile;
2-amino-8-ethyl-4-methyl-6-pyrimidin-5-ylpyrido[2,3-d]pyrimidin-7(8H)-one;
2-amino-8-ethyl-6-(1H-imidazol-5-yl)-4-methylpyrido[2,3-d]pyrimidin-7(8H)-one;
2-amino-8-ethyl-4-methyl-6-(1H-1,2,3-triazol-5-yl)pyrido[2,3-d]pyrimidin-7(8H)-one;
2-amino-8-ethyl-4-methyl-6-(1H-pyrazol-4-yl)pyrido[2,3-d]pyrimidin-7(8H)-one;
2-amino-8-ethyl-4-methyl-6-(1,3-thiazol-2-yl)pyrido[2,3-d]pyrimidin-7(8H)-one;
2-amino-8-ethyl-4-methyl-6-(1H-tetrazol-5-yl)pyrido[2,3-d]pyrimidin-7(8H)-one;
2-amino-8-ethyl-4-methyl-6-(1-methyl-1H-pyrrol-2-yl)pyrido[2,3-d]pyrimidin-7(8H)-one;
2-amino-4,8-diethyl-6-(1H-pyrazol-5-yl)pyrido[2,3-d]pyrimidin-7(8H)-one; and
2-amino-8-cyclopentyl-4-methyl-6-(1,3-thiazol-5-yl)pyrido[2,3-d]pyrimidin-7(8H)-one.

In another embodiment, the compound of formula IA is 2-amino-8-ethyl-4-methyl-6-(1H-pyrazol-5-yl)pyrido[2,3-d]pyrimidin-7(8H)-one (Compound A) or a pharmaceutically acceptable salt thereof.

### General Administration

In one aspect, the invention provides pharmaceutical compositions comprising an inhibitor of the PI3Ks and mTOR of formula I and a pharmaceutically acceptable carrier, excipient, or diluent. In certain other specific embodiments, administration is by the oral route. Administration of the compounds of formula I, or their pharmaceutically acceptable salts, in pure form or in an appropriate pharmaceutical composition as described herein, can be carried out via any of the accepted modes of administration or agents for serving similar utilities. Thus, the compound of formula I can be administered in the same or separate vehicles. Administration can be, for example, orally, nasally, parenterally (intravenous, intramuscular, or subcutaneous), topically, transdermally, intravaginally, intravesically, intracisternally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions can include a conventional pharmaceutical carrier or excipient and a compound of formula I as the/an active agent, optionally in combination with another agent, and, in addition, may include carriers and adjuvants, and so on.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

The choice of formulation depends on various factors such as the mode of drug administration (e.g., for oral administration, formulations in the form of tablets, pills, or capsules) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alginates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol, and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil, and sesame oil, glycerol, tetrahydrofurfuryl alcohol, polyethyleneglycols, and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol, and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1 percent to about 99 percent by weight of a compound(s) of the invention, or a pharmaceutically acceptable salt thereof, and 99 percent to 1 percent by weight of a suitable pharmaceutical excipient. In one example, the composition will be between about 5 percent and about 75 percent by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain an effective amount of a compound of the invention, or a pharmaceutically acceptable salt thereof, for treatment of a disease-state in accordance with the teachings of this invention.

In the pharmaceutical compositions disclosed herein, the compounds of formula I, or their pharmaceutically acceptable salts or solvates, are administered in an effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of formula I can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compounds of this invention within the dosage range described above and the other pharmaceutically active agent(s) within approved dosage ranges. Compounds of formula I may alternatively be used sequentially with known pharmaceutically acceptable agent(s) when a combination formulation is inappropriate.

In some embodiments, the effective amount produces at least one therapeutic effect selected from the group consisting of reduction in size of a tumor, reduction in metastasis, complete remission, partial remission, stable disease, increase in overall response rate, or a pathologic complete response. In some embodiments, the effective amount produces an improved clinical benefit rate (CBR=CR (complete remission)+PR (partial remission)+SD (stable disease)≧6 months) as compared to other treatments. In some embodiments, the improvement of clinical benefit rate is about 20 percent or higher. In some embodiments, the improvement of clinical benefit rate is at least about 20 percent, 30 percent, 40 percent, 50 percent, 60 percent, 70 percent, 80 percent, or more. In some embodiments, the therapeutic effect is an increase in overall response rate. In some embodiments, the increase in overall response rate is about 10 percent, 20 percent, 30 percent, 40 percent, 50 percent, 60 percent, 70 percent, 80 percent, or more.

In some embodiments, a comparable clinical benefit rate (CBR=CR (complete remission)+PR (partial remission)+SD (stable disease)≧6 dosing cycles) is obtained with treatment of a) Compound A or a pharmaceutically acceptable salt thereof, as compared to other treatments administered without Compound A. In some embodiments, the improvement of clinical benefit rate is at least about 20 percent. In some embodiments, the improvement of clinical benefit rate is at least about 30 percent. In some embodiments, the improvement of clinical benefit rate is at least about 40 percent. In some embodiments, the improvement of clinical benefit rate is at least about 50 percent. In some embodiments, the improvement of clinical benefit rate is at least about 60 percent. In some embodiments, the improvement of clinical benefit rate is at least about 70 percent. In some embodiments, the improvement of clinical benefit rate is at least about 80 percent.

In some embodiments, a comparable clinical benefit rate (CBR=CR (complete remission)+PR (partial remission)+SD (stable disease)≧6 months) is obtained with treatment of a) Compound A or a pharmaceutically acceptable salt thereof as compared to other treatments without Compound A. In some embodiments, the improvement of clinical benefit rate is at least about 20 percent. In some embodiments, the improvement of clinical benefit rate is at least about 30 percent. In some embodiments, the improvement of clinical benefit rate is at least about 40 percent. In some embodiments, the improvement of clinical benefit rate is at least about 50 percent. In some embodiments, the improvement of clinical benefit rate is at least about 60 percent. In some embodiments, the improvement of clinical benefit rate is at least about 70 percent. In some embodiments, the improvement of clinical benefit rate is at least about 80 percent.

In another aspect, methods are provided herein for assessing the therapeutic effect of Compound A for the treatment of patients having mantle cell lymphoma, follicular lymphoma, or chronic lymphocytic leukemia/small lymphocytic lymphoma, comprising determining and comparing the pre-treatment and post-treatment levels of at least one biomarker in a patient's blood or tissue sample. A difference in the level of a biomarker in a patient's blood or tissue compared to a control may provide an indication of the clinical benefit of Compound A. For instance, an increase or decrease in the level of a biomarker in a patient may be indicative of a clinical benefit. The methods may provide quantitative results which allow the progress of treatment with Compound A to be continuously monitored, for instance, to determine whether the condition is improving or worsening.

In some embodiments, the biomarker can be a circulating protein marker as found in plasma, such as VEGF-A, PIGF, glucose, insulin, circulating and tissue micro-RNAs; circulating plasma DNA; mutations in genes encoding the PI3K catalytic subunit; and target-specific DNA markers (peripheral blood mononuclear cells), circulating cancer cells, plasma DNA, and cancer samples). In other embodiments, the biomarker can be a cancer DNA marker, such as mutations in genes encoding PI3K catalytic and/or regulatory subunits; or silencing or activating complementing events (for example, PTEN, KRAS, BRAF, LKB-1). In other embodiments, the marker can be a non-cancer DNA marker, such as provided by or relating to SNP analysis to relate genotype to safety, tolerability, pharmacokinetics, pharmacodynamics, and potential efficacy of Compound A; phosphorylation markers (cancer tissue samples, peripheral blood mononuclear cells, and circulating cancer cells); phospho-receptors (pEGFR and pMET); MAPK pathway (pMEK and pERK); PI3K pathway (pAKT [two epitopes], pGSK3β, pPRAS40, p4EBP1, pFKHR, pNF-kB, pBAD, and pCaspase 9). Methods for assessing concentration of markers in blood or tissue samples are readily available to the skilled practitioner.

### General Synthesis

Compounds of this invention can be made by the synthetic procedures described below. The starting materials and reagents used in preparing these compounds are either available from commercial suppliers such as Aldrich Chemical Co. (Milwaukee, Wis.) or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of formula I that may be prepared through the syntheses described herein may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention. Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention. In particular, imidazol-5-yl and pyrazol-5-yl each can also exist in their respective tautomeric forms imidazol-4-yl and pyrazol-3-yl. Regardless of which structure or which terminology is used, each tautomer is included within the scope of the Invention.

The present invention also includes N-oxide derivatives and protected derivatives of compounds of formula I. For example, when compounds of formula I contain an oxidizable nitrogen atom, the nitrogen atom can be converted to an N-oxide by methods well known in the art. When compounds of formula I contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of formula I can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts, or solvents, or by converting one enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. For specific examples, see M. Barvian et al., J. Med. Chem. 2000, 43, 4606-4616; S. N. VanderWei et al., J. Med. Chem. 2005, 48, 2371-2387; P. L. Toogood et al., J. Med. Chem. 2005, 48, 2388-2406; J. Kasparec et al., Tetrahedron Letters 2003, 44, 4567-4570; and references cited therein. See also U.S. Pre-grant publication US2004/0009993 A1 (M. Angiolini et al.), which is incorporated herein by reference, and references cited therein. The following examples illustrate but do not limit the invention. All references cited herein are incorporated by reference in their entirety.

A compound of the invention, wherein R¹ is optionally substituted alkyl, R² is hydrogen or optionally substituted alkyl, R⁴ is methyl or ethyl, R⁶ is phenyl or heteroaryl each of which is optionally substituted with 1, 2, 3, 4, or 5 R⁹ groups (as defined in the Summary of the Invention), and R² is hydrogen, can be prepared according to Scheme 1.

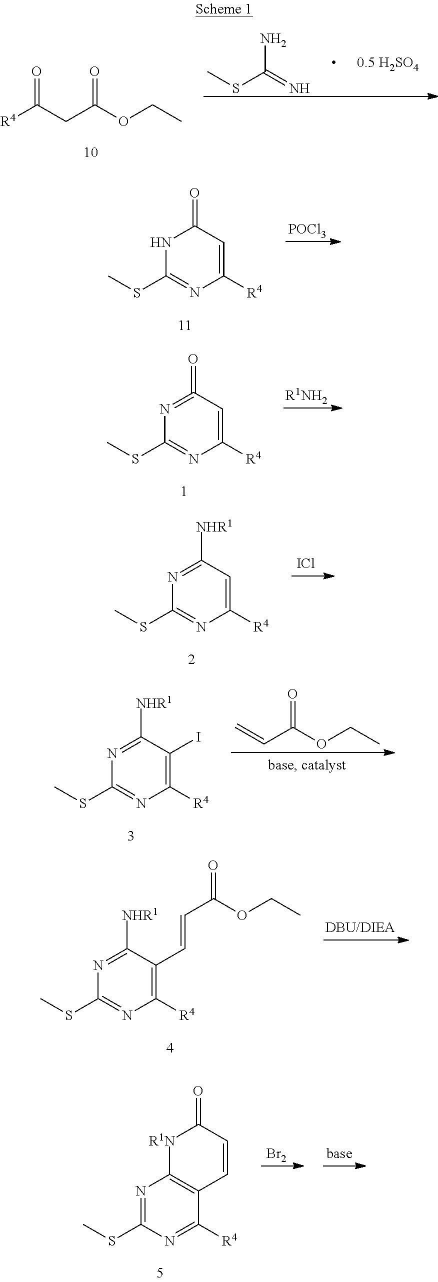

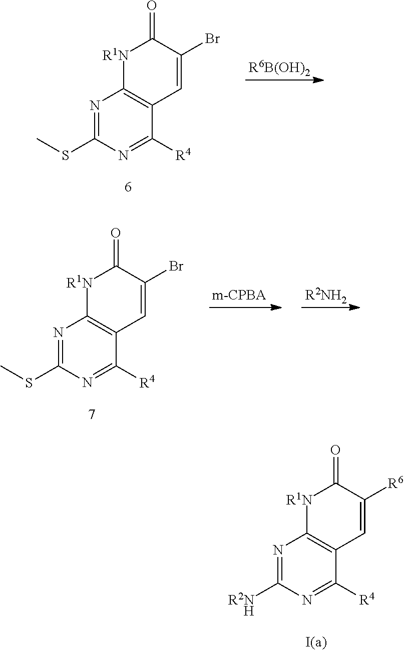

To a solution of commercially available 2-methyl-2-thiopseudourea sulfate in a solvent such as water is added a base such as sodium carbonate and an intermediate of formula 10 at room temperature. The reaction mixture is stirred for overnight or less. After neutralizing, 11 is collected through filtration and followed by drying under vacuum. 11 is then treated with POCl₃ and the reaction is heated to reflux for approximately 2 hours and then concentrated under vacuum to dryness. 1 can be used directly in the next reaction without further purification.

An intermediate of formula 2 is prepared by reacting an intermediate of formula 1 with a primary amine R¹NH₂ in a solvent such as water and with heating. 2 is then treated with iodine monochloride in a solvent such as methanol at around 0° C. and allowed to react for approximately overnight or less as needed for the reaction to go to completion to form 3. After completion the residue is triturated with acetone. The intermediate 3 is then reacted in a solvent, such as DMA, with ethyl acrylate in the presence of a base, such as triethylamine, and in the presence of a catalyst, such as Pd(OAc)₂, and (+)BINAP. The reaction is heated to approximately 100° C. and allowed to react for approximately overnight or less as needed for the reaction to go to completion to form 4. 4 is then optionally purified by column chromatography.

5 is prepared by treating 4 with DBU in the presence of a base such as DIPEA at room temperature. The reaction mixture is then heated to reflux and reacted for approximately 15 hours. After evaporation of solvent, the residue is triturated with acetone and collected by filtration to yield 5.

6 is prepared by reacting 5 with a brominating agent such as Br₂ in a solvent such as DCM at room temperature. The reaction mixture is then stirred for approximately overnight. The resulting product is filtered and then suspended in a solvent such as DCM and treated with a base such as triethylamine. The mixture is then washed with water and dried over a drying agent such as Na₂SO₄ to yield 6.

A Suzuki coupling is then performed using 6 and a boronic acid (or ester) of formula R⁶B(OH)₂ in a solvent(s) such as a DME-H₂O mixture in the presence of a catalyst such as Pd(dpppf) and a base such as triethylamine at room temperature. The reaction mixture is heated to reflux for approximately 4 hours-. After cooling to room temperature, the reaction mixture is partitioned with water and ethyl acetate. After separation, the organic layer is dried over a drying agent such as Na₂SO₄ to yield 7.

The methylthio group of 7 is then oxidized with m-CPBA in a solvent such as DCM at room temperature with stirring for approximately 4 hour. After removal of the solvent under reduced pressure, the product is treated with an amine of formula R²NH₂ in a solvent such as dioxane and stirred at room temperature for approximately overnight to yield a compound of formula I.

Alternatively, a compound of formula I where R¹ is optionally substituted alkyl, R⁴ is methyl or ethyl, R⁶ is phenyl or heteroaryl each of which is optionally substituted with 1, 2, 3, 4, or 5 R⁹ groups (as defined in the Summary of the Invention), and R² is hydrogen can be prepared according to Scheme 2.

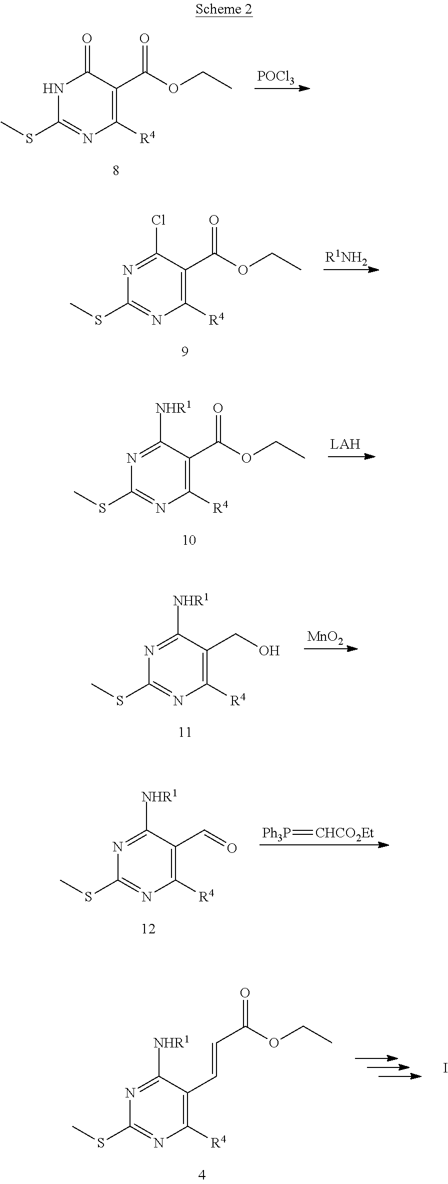

An intermediate of formula 9 is prepared by reacting an intermediate of formula 8 with neat POCl₃ and heating. 9 is then treated with a primary amine R¹NH₂ in a solvent such as water or THF and triethylamine at 0° C. to form 10. After removal of the solvent under reduced pressure, the intermediate 10 is then reacted with lithium aluminum hydride in a solvent such as THF at 0° C. After quenching and aqueous workup, solvent removal provided crystalline 11 without further purification. Treatment of 11 with manganese (II) dioxide in a solvent such as methylene chloride or chloroform at room temperature provided aldehyde 12 upon filtration and solvent removal. A Wittig reaction with aldehyde 12 can be employed with (carbethoxymethylene)triphenylphosphorane in refluxing THF to provide the common intermediate 4. 4 can then be used to prepare a compound of formula I using the procedures described in Scheme 1.

A compound of the invention where R¹ is optionally substituted alkyl, R⁴ is methyl or ethyl, R⁶ is phenyl or heteroaryl each of which is optionally substituted with 1, 2, 3, 4, or 5 R⁹ groups (as defined in the Summary of the Invention), and R² is hydrogen can be prepared according to Scheme 3.

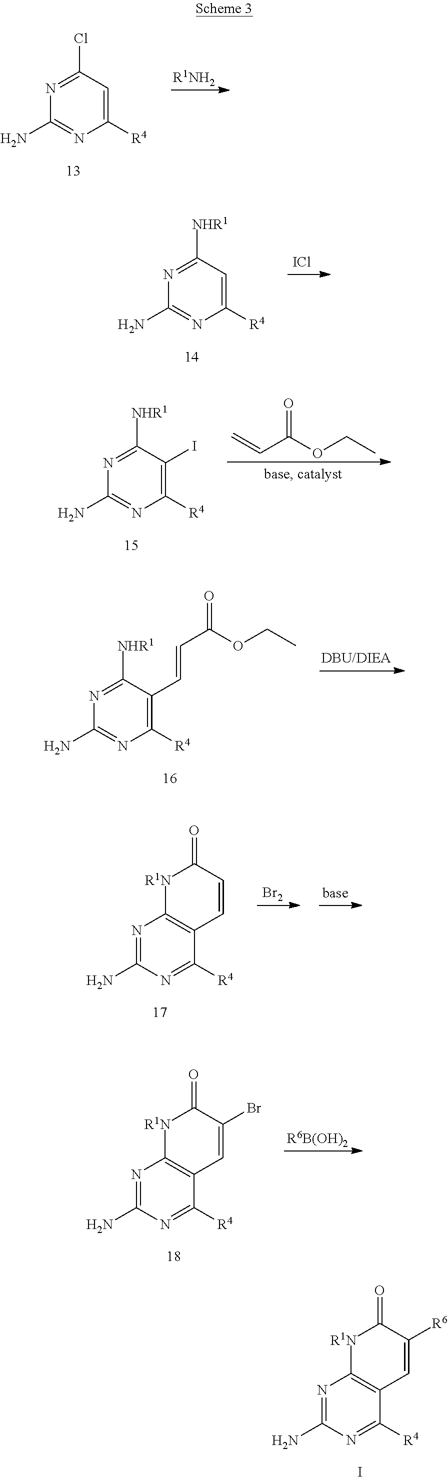

An intermediate of formula 14 is prepared by reacting an intermediate of formula 13 with a primary amine R¹NH₂ in a solvent such as water and with heating. 14 is then treated with iodine monochloride in a solvent such as methanol at around 0° C. and allowed to react for approximately overnight or less as needed for the reaction to go to completion to form 15. After completion the residue is triturated with acetone. The intermediate 15 is then reacted in a solvent, such as DMA, with ethyl acrylate in the presence of a base, such as triethylamine, and in the presence of a catalyst, such as Pd(OAc)₂, and (+)BINAP. The reaction is heated to approximately 100° C. and allowed to react for approximately overnight or less as needed for the reaction to go to completion to form 16. 16 is then optionally purified by column chromatography. A compound of formula I can then be prepared from 16 by using the same reaction conditions as described in Scheme 1 (starting at the point of the preparation of 5 from 4).

A compound of the invention where R¹ is optionally substituted alkyl, R⁴ is methyl or ethyl, R⁶ is phenyl or heteroaryl each of which is optionally substituted with 1, 2, 3, 4, or 5 R⁹ groups (as defined in the Summary of the Invention), and R² is hydrogen can alternatively be prepared according to Scheme 4.

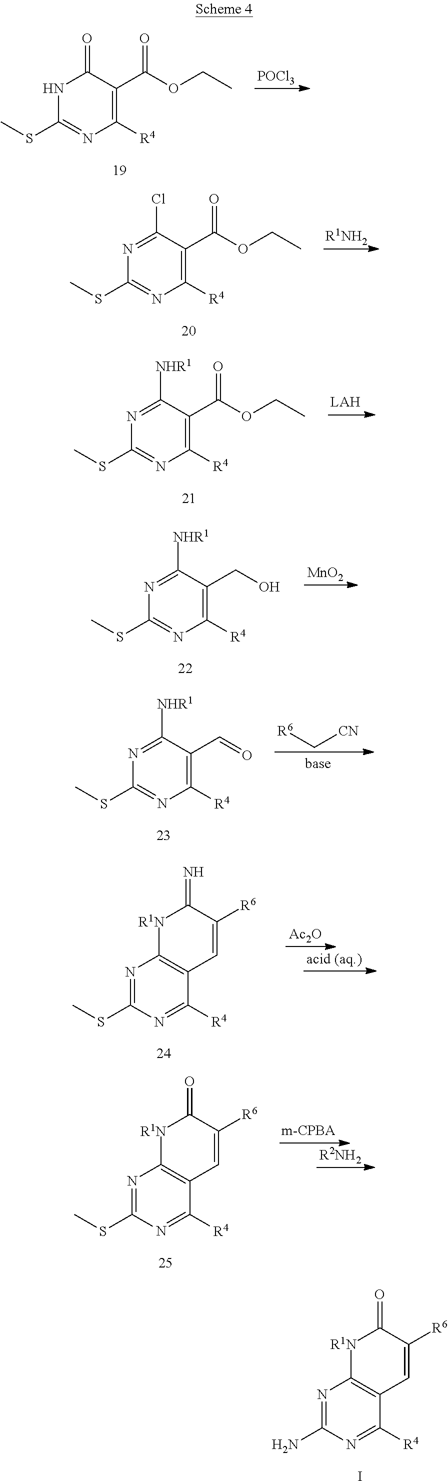

An intermediate of formula 20 is prepared by reacting an intermediate of formula 19 with neat POCl₃ and heating. 20 is then treated with a primary amine R¹NH₂ in a solvent such as water or THF and triethylamine at 0° C. to form 21. After removal of the solvent under reduced pressure, the intermediate 21 is then reacted with lithium aluminum hydride in a solvent such as THF at 0° C. After quenching and aqueous workup, solvent removal provides crystalline 22 without further purification. Treatment of 22 with manganese (II) dioxide in a solvent such as methylene chloride or chloroform at room temperature provides aldehyde 23 upon filtration and solvent removal. A Knovenegal-type condensation with 23 and an arylacetonitrile in the presence of a base such as potassium carbonate or sodium hydroxide in a protic solvent provides the cyclized imine 24. Acetylation of the imine with acetic anhydride is required prior to hydrolysis, which takes place in the presence of aqueous acid and heating to afford 25. Subsequently, 25 can be oxidized to the corresponding sulfone with m-CPBA at room temperature and displaced with ammonium to provide I.

The synthesis of specific compounds is described in WO2007 0444813 which is hereby incorporated by reference in its entirety.

### EXAMPLES

Suitable in vitro assays for measuring PI3K activity and the inhibition thereof by compounds are known in the art. Compounds of formula I have been tested using one or more of the assays described in Biological Examples 1 and 2. Assays for measurement of in vitro efficacy in the treatment of cancer are known in the art (see also Biological Examples, Example 3, 4, and 5 infra).

### Example 1

### Phase 1 Dose-Escalation Study to Assess the Safety, Pharmacokinetics (PK) and Pharmacodynamics of Compound A Administered Orally to Patients with Advanced Malignancies

A Phase I, non-randomized, open-label, dose-escalation study was conducted for Compound A using two dosing schedules to evaluate the safety, PK, and pharmacodynamics of Compound A in subjects with solid tumors or lymphoma.

Patients were treated with a conventional ‘3+3’ dose-escalation design with cohorts of three to six subjects for up to nine different dose levels on each dosing schedule.

Patients received 5, 10 or 50 mg of Compound A either twice daily (bid) or daily (qd) for 28-day cycles. Pharmacokinetics and pharmacodynamic analyses were performed, and tumor response was assessed every 8 weeks.

### Results

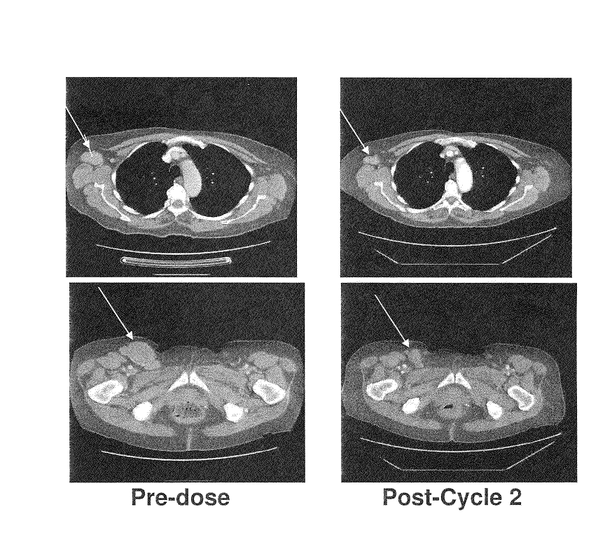

96 patients were dosed with Compound A, including 83 patients with solid tumors and 13 with lymphoma. 52 patients were dosed on a bid regimen (30-240 mg/day) and 31 patients on a qd regimen (70-100 mg/day, and 13 patients with lymphoma on a 50 mg bid regimen). On the bid schedule, 25 subjects were treated at the established the maximum tolerated dose (MTD) of 50 mg bid. The maximum administered dose (MAD) is 120 mg bid. On the qd schedule, the MAD is 100 mg, and the established MTD is 90 mg. The most common related adverse events (greater than 10 percent of patients) were nausea, diarrhea, anorexia, elevated liver enzymes, skin and subcutaneous disorders, and vomiting. Grade≧3 AST/ALT elevation occurred in four patients (three patients initially dosed at 120 mg bid and one patient initially dosed at 50 mg bid). Compound A exposure increased with increasing doses on bid and qd schedules. Median tₘₐₓ was 1-3 hours post-dose. Mean t_{1/2,z} ranged from 3 to 9 hours at steady-state. Robust pharmacodynamic modulation of PI3K and ERK pathway signaling was evident in tumors and surrogate tissues following repeat-dose administration of Compound A. For example, post-dose reductions in pAKT-T308 (57-71 percent), p4EBP1 (62-80 percent), and pERK (53-80 percent) were seen in paired biopsies of diverse solid tumors from five patients administered Compound A at 50 mg bid. Eleven patients were on study for ≧16 weeks and seven patients on treatment for ≧24 weeks. One patient with mantle cell lymphoma (MCL) exhibited a partial response and was treated for 12 cycles (FIG. 1). The patient, a 75 year old female with stage III MCL (RP LN, bone marrow negative), was diagnosed in April 2009 and initially responded to 6 cycles of R—CHOP between May-August 2009. Maintenance treatment of rituximab following R—CHOP had allowed progression.

### CONCLUSIONS

An MTD for single-agent Compound A was identified as 50 mg bid and 90 and qd. Activity was observed in solid tumor patients with prolonged disease stabilization and in a lymphoma subject (one partial response in MCL). Compound A exhibited potent pharmacodynamic activity in solid tumors and surrogate tissues at generally well-tolerated doses.

### Example 2

### Phase 1 Dose-Expansion Cohort Study of the Safety, Pharmacokinetics and Pharmacodynamics of Compound A, an Orally Administered PI3K/mTOR Inhibitor, in Patients with Lymphoma

The following phase 1 study was designed to evaluate the safety and pharmacokinetics of Compound A administered as a continuous daily dosing regimen in an expansion cohort of subjects with relapsed or refractory lymphoma.

As indicated by the results of Example 5A, Compound A was generally well-tolerated with the maximum tolerated doses of 50 mg twice daily or 90 mg once daily. Plasma exposure levels of Compound A were found to increase with increasing dose. At the maximum tolerated dose, the daily and twice daily dosing regimens appeared to yield similar average plasma exposure at steady state.

### Patients and Methods

Study Design

This was a Phase 1, open-label, non-randomized trial of Compound A given orally as a single agent in subjects with lymphoma. The maximum tolerated dose expansion cohort planned enrollment was at least 15 subjects.

Objectives

The primary objective was to determine the safety and tolerability of Compound A administered orally, twice daily, as a continuous daily dosing regimen to subjects with relapsed or refractory lymphoma.

The secondary objectives were to determine (a) the plasma pharmacokinetics of Compound A continuous daily dosing; and (b) the pharmacodynamic effects of Compound A on tumor tissue.

The exploratory objectives were to determine (a) the pharmacodynamic effects of Compound A in subjects with lymphoma; (b) the preliminary efficacy data (response rate) for Compound A; and (c) the long-term safety and tolerability of Compound A after repeated twice daily administration.

Key Eligibility Criteria

The key eligibility include the following:
Histologically confirmed diagnosis of relapsed or refractory lymphoma
Measureable disease
Adequate bone marrow function defined as:
ANC≧1000/mm3 (chronic lymphocytic leukemia with ANC≧500/mm3)
Platelets≧30,000/mm3
Hemoglobin≧8 g/dL
Availability of archival or fresh tumor tissue
No prior therapy with a selective PI3K inhibitor
Written informed consent.

### Results

Sixteen patients were enrolled in the lymphoma cohort. The preliminary data is presented as of the cut-off date of Sep. 1, 2011. Baseline characteristics of the patients are presented in Table 2.1.

TABLE 2.1

| Characteristic | N = 16 |
|---|---|
| Age in years, median (range) | 69.5 (20-90) |
| Sex, n (%) | |
| Male | 12 (75) |
| Female | 4 (25) |
| Lymphoma type, n (%) | |
| Anaplastic large cell (T and null cell) | 1 (6) |
| Diffuse large B-cell | 2 (13) |
| Follicular grade 1-2 | 5 (31) |
| Hodgkin | 1 (6) |
| Mantle cell | 6 (38) |
| Transformed | 1 (6) |
| Stage, n (%) | |
| II | 2 (13) |
| III | 3 (19) |
| IV | 11 (70) |
| ECOG performance status, n (%) | |
| 0 | 4 (25) |
| 1 | 8 (50) |
| 2 | 4 (25) |
| Baseline Hematologic Parameters, | |
| median (range) | |
| Hemoglobin (G/L) | 115 (93-154) |
| Platelets (109/L) | 261.50 (64.90-327.30) |
| Absolute lymphocyte count (109/L) | 0.85 (.21-135.84) |
| No. of prior anticancer regimens within | 3.5 (1-8) |
| 5 years, median (range) | |

The most common adverse events experienced among the sixteen test subjects are summarized in Table 2.2.

TABLE 2.2

| n (%) | All Adverse Events^{a} | | Related Adverse Events^{a} | |
|---|---|---|---|---|
| Adverse Event | All Grades | Grade ≧ 3 | All Grades | Grade ≧ 3 |
| Hematologic | | | | |
| Anemia | 3 (19) | 2 (13) | 1 (6) | 0 |
| Non-Hematologic | | | | |
| Diarrhea | 6 (38) | 0 | 3 (19) | 0 |
| Nausea | 5 (31) | 0 | 5 (31) | 0 |
| AST increased | 4 (25) | 0 | 3 (19) | 0 |
| Pyrexia | 4 (25) | 0 | 0 | 0 |
| ALT increased | 3 (19) | 2 (13) | 3 (19) | 2 (13) |
| Asthenia | 3 (19) | 2 (13) | 0 | 0 |
| Fatigue | 3 (19) | 1 (6) | 1 (6) | 0 |
| Creatinine increased | 3 (19) | 0 | 0 | 0 |
| Hyperglycemia | 3 (19) | 0 | 1 (6) | 0 |

^{a}MedDRA v. 12.1 Preferred Terms and NCI-CTCAE v. 3.0 grading

Study treatment details for the sixteen subjects are summarized in Table 2.3.

TABLE 2.3

| | |
|---|---|
| Median duration of therapy, weeks (range) | 9 (1-66) |
| Patients requiring dose modification due to toxicity, n (%) | 12 (75) |
| Patients requiring dose modification due to treatment- | 5 (31) |
| related toxicities, n (%) | |
| AST/ALT increase | 2 (40) |
| Nausea | 2 (40) |
| Amylase increase | 1 (20) |
| Discontinuations of treatment, n (%) | 14 (88) |
| Adverse event | 2 (13) |
| Progressive disease | 8 (50) |
| Death (unrelated to study drug) | 1 (6) |
| Other reasons | 3 (19) |

Table 2.4 summarizes the best response in the thirteen subjects evaluated for efficacy.

TABLE 2.4
Best Response in Subjects Evaluated for Efficacy.^{a}

| | | Complete | Partial | Stable | Progressive |
|---|---|---|---|---|---|
| Malignancy | n | Response | Response | Disease | Disease |
| Mantle Cell | 5 | | 1^{b} | 3 c | 1 |
| Lymphoma | | | | | |
| Transformed | 1 | | 1 d | | |
| Lymphoma | | | | | |
| Diffuse Large B-cell | 2 | | 1 | | 1 |
| Lymphoma | | | | | |
| Follicular | 4 | | | 3 | 1 |
| Lymphoma | | | | | |
| Hodgkin′s | 1 | | | 1 | |
| Lymphoma | | | | | |

^{a}Subjects with disease assessments after completion of 2 cycles (8 weeks) of Compound A administered twice daily as a continuous daily dosing regimen.
^{b}Partial response achieved after cycle 2, currently in cycle 17.
c One patient had maintained stable disease through cycle 14.
d Partial response achieved after cycle 5, currently in cycle 7.

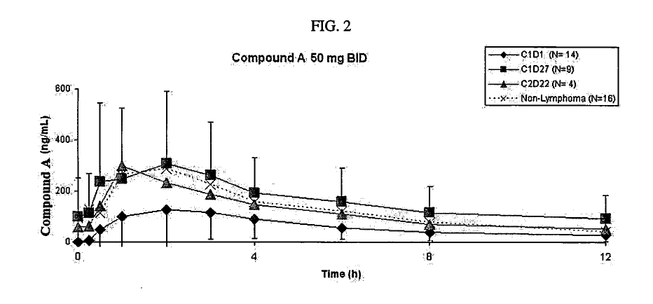

FIG. 2 depicts the mean (SD plasma concentration of Compound A in cycle 1 on Days 1 and 27 (C1D1, D1D27), and during cycle 2 on day 22 (C2D22) after daily treatment with oral Compound A 50 mg twice daily (BID). FIG. 2 indicates that drug exposure in the lymphoma patients who were enrolled in the study was similar to that seen previously in patients with solid tumors after 50 mg BID daily doses (mean concentrations shown in dashed line). The accumulation (AUCtau) on day 27 was approximately 3-fold, and the AUCtau ratio of C2D22 versus C1D27 (n=4) was 0.91 (64.4%).

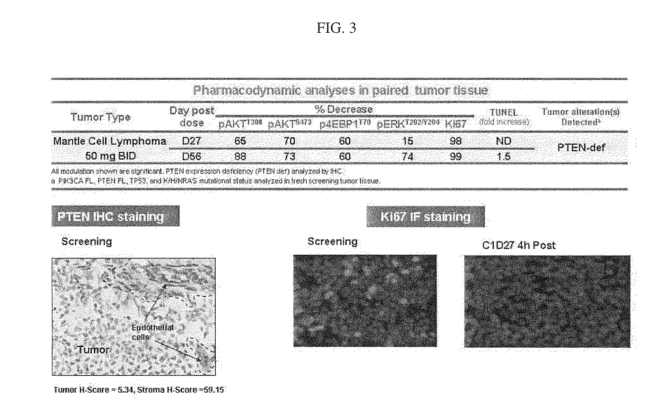

The data in Table 2.5 demonstrates the ability of Compound A to inhibit the PI3K and MAPK pathways in a mantle cell lymphoma tumor. The results indicate robust inhibition of the PI3K and MAPK pathways, as well as near complete inhibition of Ki67 proliferation. This finding is also reflected in FIG. 3, which depicts PTEN IHC and Ki67 IF staining of lymphoma tumor cells treated with Compound A. PTEN expression was evaluated by immunohistochemistry in formalin fixed, paraffin-embedded tissue sections FFPE (5 micron) with anti-PTEN antibody (NCL-PTEN, clone 28H6, diluted 1/300, Novocastra Laboratories Ltd) utilizing standardized procedures. Cryopreserved tumor biopsy samples were serially sectioned at 5 microns and converted in FFPE sections.

TABLE 2.5
Pharmacodynamic analyses in paired tumor tissue

| | | | | | | | TUNEL | Tumor |
|---|---|---|---|---|---|---|---|---|
| Tumor | Day post | % Decrease | | | | | (fold | alteration(s) |
| Type | dose | pAKTT308 | pAKTS473 | p4EBP1T70 | pERKT202/Y204 | Ki67 | increase) | Detecteda |
| Mantle Cell | D 27 | 65 | 70 | 60 | 15 | 98 | ND | PTEN-def |
| Lymphoma | D 56 | 88 | 73 | 60 | 74 | 99 | 1.5 | |
| 50 mg BID | | | | | | | | |

All modulation shown are significant. PTEN expression deficiency (PTEN def) analyzed by IHC.
a PIK3CA FL, PTEN FL, TP53, and K/H/NRAS mutational status analyzed in fresh screening tumor tissue.

### Summary

Compound A is a dual PI3K/mTor inhibitor that is well tolerated in subjects with lymphoma. The pharmacokinetic profile of Compound A in patients with lymphoma is consistent with that seen in patients with solid tumors. Robust pharmacodynamic modulation of PI3K and ERK pathway signaling was evident in PTEN deficient mantle cell lymphoma patients. MAPK pathway inhibition appears to be due to an indirect impact. Near complete inhibition of proliferation was observed, as shown by Ki67 staining.

### Example 3

### Treatment of Lymphoproliferative Malignancies with Compound A

This is a 2-stage Phase 2, nonrandomized, open-label, multi-center study. Patients will enroll in 1 of 3 groups: Group 1 will enroll patients with relapsed or refractory (R/R) MCL, Group 2 will enroll patients with R/R Grade 1, Grade 2, or Grade 3a FL, Group 3 will enroll patients with R/R CLL/SLL, and Group 4 will enroll patients with R/R DLBCL (this last group will use a 1-stage design). Objective response rate is the standard primary efficacy endpoint for Phase 2 studies according to FDA and Committee for Medicinal Products for Human Use (CHMP) guidelines. Although surrogate endpoints such as response rate may not be the best measure in evaluating targeted therapies, evidence that a drug is able to produce tumor shrinkage is considered adequate evidence of antitumor activity to indicate that a new drug deserves further evaluation.

### Study Objectives

The primary objective of this study is to evaluate the efficacy of Compound A in patients with one of the following relapsed or refractory lymphoma or leukemia subtypes: MCL, FL, CLL/SLL, or DLBCL. The secondary objectives of this study are: (i) to assess the duration of response, progression free survival (PFS) and proportion of patients with PFS at 6 months (24 weeks) in patients with either MCL, FL, CLL/SLL, or DLBCL treated with Compound A; (ii) to evaluate the safety and tolerability of Compound A in patients with MCL, FL, CLL/SLL, or DLBCL; and (iii) to further characterize the plasma pharmacokinetics (PK) of Compound A in patients with MCL, FL, CLL/SLL, or DLBCL. The exploratory objectives are (i) to assess the pharmacodynamic effects of Compound A in patients with MCL, FL, CLL/SLL, or DLBCL; and (ii) to define predictive markers of response and/or resistance to Compound A based on molecular profiling of cancer tissue.

### Study Design

This is a multicenter, multinational, nonrandomized, open-label, 2-stage, Phase 2 clinical trial of Compound A at 50 mg orally twice daily (bid) for a 28-day dosing cycle in patients with relapsed or refractory (R/R) MCL, FL, CLL/SLL, or DLBCL having failed at least 2 standard treatment regimens. Patients will enroll into one of three groups based on disease:

Group 1: R/R MCL

Group 2: R/R Grade 1, 2, or 3a FL

Group 3: R/R CLL or SLL

Group 4: R/R DLBCL

Simon's minimax 2-stage design will be used to determine whether the drug is potentially efficacious to warrant further study in 1 or more of the disease groups studied; Group 4 (DLBCL) will use a 1-stage design. Objective responses will be assessed by the Investigator according to the International Working Group for Lymphoma (IWL) and International Working Group on Chronic Lymphocytic Leukemia (IWCLL) criteria.

### Study Population

### Main Inclusion Criteria:

Histologically and phenotypically confirmed mantle cell lymphoma (MCL) which has relapsed or has been refractory to at least 2 but not more than 4 prior antineoplastic therapies.
Histologically or cytologically confirmed Grade 1, 2, or 3a follicular lymphoma (FL) which has relapsed or has been refractory to at least 2 but not more than 6 prior antineoplastic therapies.
Histologically or cytologically confirmed chronic lymphocytic leukemia (CLL) chronic lymphocytic leukemia (CLL) which has relapsed or has been refractory to at least 2 but not more than 6 prior antineoplastic therapies and requires treatment according to the IWCLL criteria.
Histologically or cytologically confirmed small lymphocytic lymphoma (SLL) which has relapsed or has been refractory to at least 2 but not more than 6 prior antineoplastic therapies.
   Refractory disease is defined as unresponsive to a standard regimen or progressing within 6 months of completing a standard regimen.
Histologically or cytologically confirmed Diffuse Large B-cell Lymphoma (DLBCL) which has relapsed or has been refractory to at least 2 but not more than 6 prior antineoplastic therapies.
   Refractory disease is defined as unresponsive to a standard regimen or progressing within 6 months of completing a standard regimen.
Patients with MCL, FL, SLL or DLBCL must have at least 1 target lesion measuring ≧1.5 cm in the longest transverse diameter and clearly measurable in at least 2 perpendicular dimensions, by computerized tomography (CT) (or magnetic resonance imaging [MRI] if CT scan cannot be performed) or contrast enhanced PET/CT that has not been previously irradiated or has increased in size following irradiation. Baseline 18-flouro-deoxyglucose positron emission tomography (FDG-PET) is recommended but not required.
At least 150 microns of tissue or a tissue block of the most recent archival or fresh tumor tissue is required for MCL, FL and DLBCL.
At a minimum, a peripheral blood buffy coat sample is required for CLL/SLL.

### Main Exclusion Criteria

Treatment with cytotoxic chemotherapy, biologic agents, investigational therapies within 4 weeks, or nitrosoureas or mitomycin C within 6 weeks of study enrollment.
Treatment with a small-molecule kinase inhibitor within 2 weeks, or 5 half-lives of the drug or its active metabolites (whichever is longer) of study enrollment.
Prior treatment with a PI3K, mTOR, or Akt inhibitor. Prior treatment of MCL with temsirolimus is permitted in patients enrolled from countries where it is licensed for this indication.
Radiation therapy within 2 weeks of enrollment.
Autologous stem cell transplantation within 16 weeks of enrollment.
Prior allogeneic transplantation.
Central nervous system or leptomeningeal involvement.
Positive Hepatitis B surface antigen (HBsAg) or Hepatitis C antibody (anti-HCV) serology.

### Dose Regimen

All patients will take Compound A twice daily (in the morning and evening), with a preferred interval of 12 (±1) hours between doses as described in the Study Reference Manual.

Compound A may be reduced by 1 or 2 dose levels in patients experiencing 1 or more Grade≧2 AEs regardless of causality. All Compound A related Grade≧2 transaminase elevations, intolerable Grade≧2 skin rash and Grade≧3 adverse events (AEs) require dose reduction. Patients requiring more than 2 dose level reductions will be withdrawn from study. Dose reductions are not required for any Grade tumor lysis syndrome occurring in Dosing Cycle 1 of treatment.

One level dose re-escalation is permitted if the toxicity that led to the dose reduction was Grade≦3 and does not reoccur after 1 dosing cycle of treatment at the reduced dose level.

Dosing with Compound A must not be resumed until any IMP-related toxicities have resolved Grade≦1 or baseline.

### Primary and Secondary Endpoints

The primary endpoint will be the objective response rate (ORR) as defined as the proportion of patients who experience complete response/remission (CR) or partial response/remission (PR) as defined by the International Working Group Response Criteria for malignant lymphoma (IWRC) and modified International Workshop on Chronic Lymphocytic Leukemia guidelines (IWCLL). All patients with MCL, FL, SLL or DLBCL meeting the criteria for CR must have a confirmatory FDG-PET scan no less than 6 weeks after the CR assessment. Patients with pretreatment bone marrow involvement (determined by biopsy, flow cytometry, or IHC) will be considered a PR unless CR is confirmed by bone marrow biopsy, including molecular analysis.

Main secondary endpoints will include (i) median PFS, proportion of patients with PFS at 6 months (24 weeks), duration of response; (ii) safety (AEs and laboratory parameters); and (iii) plasma concentrations of Compound A will be measured in dosing cycles 1, 3, and 6.

### Assessment Schedule

A dosing cycle is defined as 28 days of dosing with Compound A. Collection of AE data starts at the time of informed consent and will be performed every site visit while on study treatment and 30 days after the end of study treatment. Telephone safety assessments will be performed at specified intervals in between site visits. Safety assessments (AEs, vital signs, electrocardiogram [ECG], ophthalmologic examinations, laboratory tests, and concomitant medications) will be performed prior to the start of Compound A on Dosing Cycle 1, Day 1, and according to the study flowchart.

Tumor assessments will be performed at the end of dosing cycle 2 and then every 3 dosing cycles for a period of 2 years or until disease progression or withdrawal from study. Patients who continue on study beyond 2 years will have tumor assessments at a minimum of every 6 dosing cycles.

Compound A plasma concentration analysis will be performed separately for patients with MCL, FL, CLL/SLL, or DLBCL. Blood samples will be obtained at scheduled time points and if possible, whenever there is an IMP-related SAE.

Blood or processed blood, hair, and tumor tissue samples will be obtained for analyses of a variety of established and exploratory pharmacodynamic biomarkers on a defined schedule. When possible, PD sample collection will coincide with scheduled PK time points.

Optional on study tumor biopsies maybe collected from consented patients at specified time points. The maximum sampling is 3 biopsy time points, including baseline. The tumor tissues will be analyzed for biomarkers related to Compound A mechanism of action. Matched blood and hair sampling are required when optional biopsies are collected.

A blood sample will be obtained prior to the first dose of Compound A from patients who signed the optional pharmacogenetic (PGx) informed consent form. The PGx blood sample will be collected to investigate allelic variants of drug metabolizing enzyme (DME) and/or drug transporters as intrinsic factors associated with pharmacokinetic or pharmacodynamic variability of Compound A. For CLL patients, additional buccal swabs will be obtained from consented patients for genotyping analyses. PGx blood and buccal swap may also be used for genotyping and/or tumor genome sequencing analyses. Detailed instructions for PK/PD/PGx sample collection, preparation, storage, and shipping will be provided to the study sites in a separate Laboratory Manual.

### Statistical Considerations

Sample Size Determination

This study employs a Simon minimax 2-stage design with an alpha of 0.05 and power of 90 percent for each of the disease groups, based on following assumptions.

Analysis of Stage 1 will be performed within each of the disease groups when the required number of evaluable patients has been enrolled and completed 2 efficacy assessments (at the completion of Dosing Cycle 2 and Dosing Cycle 5).

Group 4 will use a 1-stage design (HO 0.1 and Ha 0.30), 33 evaluable patients will be enrolled.

### Analysis of Primary Endpoint

The primary efficacy analysis of objective response rate (ORR) will be performed within each of the disease groups when the required number of evaluable patients has been enrolled. Specifically, the data cutoff for the primary efficacy analysis will be defined as the earliest date when all patients have been followed for at least 6 months or discontinued from the study. This will be defined for each of the disease groups.

The efficacy population is defined as all registered patients who have received at least 2 dosing cycles of Compound A, and provide a baseline and at least 1 post-baseline tumor assessment. Patients who fail to have a post-baseline assessment due to early clinical progression, toxicity, or death will also be included.

A response rate for each disease group will be calculated based on proportion of patients who have an objective response (OR) in the efficacy population, with corresponding 95 percent CI.

### Analysis of Secondary Endpoints

Median progression free survival (PFS), and proportion of subjects with PFS at 6 months will be estimated using the Kaplan-Meier approach based on efficacy population. Incidences of AEs and SAEs will be tabulated by system organ class and preferred terms. Laboratory test results will be summarized.

Analysis of safety parameters, laboratory will be performed on the all-treated/safety population. The all-treated/safety population is defined as all registered patients exposed to the IMP, regardless of the amount of treatment administered.

### Duration of Study Period

The study consists of a 28-day screening phase prior to Compound A administration, then an on-treatment phase with 28-day study treatment dosing cycles, and a post treatment safety follow-up phase of about 30 days. Patients will continue to receive Compound A until a study withdrawal criterion is met, and will remain on study until the last post treatment visit or until Compound A-related toxicities have resolved or are deemed irreversible, whichever is later.

Disease assessments will be obtained in the Post treatment Period, following the original schedule or earlier, if clinically indicated, if study treatment is discontinued before documentation of progressive disease per the revised IWRC or IWCLL guidelines.

The expected enrollment period is approximately 24 months.

### Dose of Compound A

All patients will take Compound A twice daily (in the morning and evening), with a preferred interval of 12 (±1) hours between doses. Each 50-mg dose should preferably be administered as a single 50-mg dose strength capsule. Compound A will be taken with one glass (approximately 8 ounces (240 mL) of water, with no food allowed for at least 2 hours before and 1 hour after dosing; if a dose is missed it may be taken up to 4 hours after the normal dosing time. No doses outside the 4 hour window should be given or made up at a future time. Extra doses should not be administered if the patient vomits after taking Compound A. Patients may take other concomitant medications (except gastric pH altering medications with water at the same time that Compound A is administered). Investigational medicinal product will be administered at the study site at specific protocol-defined visits; other doses will be self-administered. Investigational medicinal product will be distributed to patients at study visits and a dispensation record will be maintained. At follow-up visits, remaining Compound A will be counted, and treatment compliance documented.

### Dose Delays/Modifications

Patients are monitored for adverse events (AE)s while enrolled in the study and are instructed to notify their physician as soon as possible at the sign of any new or worsening AEs. As a general approach, it is suggested that all AEs be treated with supportive care when possible at the earliest signs of toxicity.

Re-Escalation after Reductions for Toxicity

Depending on the severity of the AE, a patient's dose level may be re-escalated one dose level once the AE has subsided.

### Pharmacodynamic Analysis

Cancer tissues, hair, and blood including peripheral blood buffy coats will be collected for pharmacodynamic analysis with the agreement of the Investigator and Sponsor, and with the patient's consent.

Studies may include investigation of the impact of target mutations (PI3K catalytic and/or regulatory subunits) preexisting in the patient's cancer on response, fluctuation of plasma levels of pathway-relevant proteins (for example, VEGF-A, glucose, and insulin), drug-induced changes in phosphorylation of signal transduction proteins and lipids (for example, pAKT, pERK, pGSK3β, and PIP3), as well as assessment of the contribution of complementing genetic changes in target modulators (for example, PTEN, KRAS, and LKB-1) on efficacy.

Representative candidate biomarkers that may be analyzed in each specimen type collected for pharmacodynamic analyses is provided below:

### Circulating Protein Markers (Plasma)

VEGF-A, PIGF, glucose, and insulin.
Circulating and tissue micro-RNAs.
Circulating plasma DNA.
Mutations in genes encoding PI3K catalytic subunit.
Target-specific DNA markers (peripheral blood mononuclear cells), circulating cancer cells, plasma DNA, and cancer samples).

### Cancer DNA

Mutations in genes encoding PI3K catalytic and/or regulatory subunits.
Silencing or activating complementing events (for example, PTEN, KRAS, BRAF, LKB-1).

### Non-Cancer DNA

SNP analysis to relate genotype to safety, tolerability, pharmacokinetics, pharmacodynamics, and potential efficacy of Compound A.
Phosphorylation markers (cancer tissue samples, peripheral blood mononuclear cells, and circulating cancer cells).
Phospho-receptors (pEGFR and pMET).
MAPK pathway (pMEK and pERK).
PI3K pathway (pAKT [two epitopes], pGSK3β, pPRAS40, p4EBP1, pFKHR, pNF-kB, pBAD, and pCaspase 9).

In assessing pharmacodynamic changes, descriptive statistics (such as a paired t-test) will be used to describe the concentration-time data and to analyze relative changes to baseline. Where appropriate, the data may be combined with data from other studies as part of a meta-analysis. The results of the pharmacodynamic analysis may be evaluated in conjunction with available pharmacokinetic and safety data.

### Response Criteria

### Mantle Cell Lymphoma, Follicular Lymphoma, Small Lymphocytic Lymphoma, Diffuse Large B-Cell Lymphoma (Revised IWG Criteria, 2007)

Complete remission (CR) requires all of the following:
Complete disappearance of all clinical evidence of disease and disease-related symptoms.
Typically FDG-avid lymphoma exists in patients who have no pretreatment PET scan or when the FDG PET scan was positive before therapy: a post treatment residual mass of any size is permitted as long as it is PET negative. Variably FDG-avid lymphomas or FDG with unknown avidity exist in patients without pretreatment PET scan, or if pretreatment PET scans were negative.
All lymph nodes and nodal masses must have regressed on CT to normal size (no more than 1.5 cm in their greatest transverse diameter for nodes greater than 1.5 cm before therapy). Previously involved nodes that were 1.1 to 1.5 cm in their long axis and greater than 1.0 cm in their short axis before treatment must have decreased to no more than 1.0 cm in their short axis after treatment.
The spleen and/or liver, if considered enlarged before therapy on the basis of a physical examination or CT scan, should not be palpable on physical examination and should be considered normal size by imaging studies, and nodules related to lymphoma should disappear. Determination of splenic involvement, however, is not always reliable, as a spleen considered normal in size still may contain lymphoma, whereas an enlarged spleen may reflect variations in anatomy, blood volume, the use of hematopoietic growth factors, or other causes rather than lymphoma.
If the bone marrow was involved by lymphoma before treatment, the infiltrate must have cleared on repeat bone marrow biopsy. The biopsy sample on which this determination is made must be adequate (with a goal of at least 20 mm unilateral core). If the sample is indeterminate by morphology, it should be negative by immunohistochemistry. A sample that is negative by immunohistochemistry but demonstrating a small population of clonal lymphocytes by flow cytometry will be considered a CR until data become available demonstrating a clear difference in patient outcome.
   Partial remission (PR) requires all of the following:
At least a 50 percent decrease in SPD of up to 6 of the largest dominant nodes or nodal masses. These nodes or masses should be selected if: they are clearly measurable in at least 2 perpendicular dimensions; they are from disparate regions of the body, and they include mediastinal and retroperitoneal areas of disease whenever these sites are involved.
No increase in the size of other nodes, liver or spleen.
Splenic and hepatic nodules must regress by at least 50 percent in their SPD or, for single nodules, in the greatest transverse diameter.
With the exception of splenic and hepatic nodules, involvement of other organs is usually evaluable and not measurable disease.
Bone marrow assessment is irrelevant for determination of a PR if the sample was positive before treatment. If positive, however, the cell type should be specified (for example, small neoplastic B cells). Patients with a CR by the previously mentioned criteria, but who have persistent morphologic bone marrow involvement, will be considered partial responders. In cases where the bone marrow was involved before therapy that resulted in a clinical CR, but with no bone marrow assessment following treatment, patients will be considered partial responders.
No new sites of disease.
Typically FDG-avid lymphoma. For patients who have no pretreatment PET scan or if the

PET scan was positive before therapy, the post treatment PET scan should be positive in at least 1 previously involved site.
Variably FDG-avid lymphomas/FDG-avidity unknown. For patients without a pretreatment PET scan, or if a pretreatment PET scan was negative, CT criteria should be used.

### Chronic Lymphocytic Leukemia

Complete remission (CR) requires all of the following:
Peripheral blood lymphocytes (evaluated by blood and differential count) below 4×10⁹ /L (4000 /μL).
Absence of significant lymphadenopathy (lymph nodes >1.5 cm in diameter) by physical examination and imaging, if baseline scans were abnormal.
No hepatomegaly or splenomegaly by physical examination and imaging, if baseline scans were abnormal.
Absence of constitutional symptoms (B symptoms).
Blood counts above the following values:
   Neutrophils more than 1.5×10⁹ /L (1500 /μL) without need for exogenous growth factors.
   Platelets more than 100×10⁹ /L (100000 /μL) without need for exogenous growth factors.
   Hemoglobin more than 110 g/L (11.0 g/dL) without red blood cell transfusion or need for exogenous erythropoietin.
Bone marrow aspirate and biopsy must have the following findings:
   Normocellular for age.
   Less than 30 percent of nucleated cells being lymphocytes.
   No B-lymphoid nodules (confirmed by IHC).
Minimal residual disease (MRD): The quality of the CR should be assessed for MRD by either 4-color flow cytometry (MRD flow) or allele-specific oligonucleotide PCR. Patients will be defined as having a CR in the absence of MRD when they have blood or marrow with less than 1 CLL cell per 10 000 leukocytes. The blood can be used for making this assessment if the patient has not received monoclonal antibodies (e.g. alemtuzumab, rituximab), within the previous 3 months. If the patient has received monoclonal antibody therapy within the past 3 months prior, the bone marrow must be used for MRD assessment.
Complete response with incomplete marrow recovery (CRi): Patients who fulfill all the criteria for a CR but who have a hypocellular marrow and persistent anemia or thrombocytopenia or neutropenia unrelated to CLL but secondary to drug toxicity. If the marrow is hypocellular, a repeat determination should be performed after 4 weeks, or until peripheral blood counts have recovered.
Nodular partial response (nPR): patients who fulfill all the criteria for CR but who have bone marrow evidence of B-lymphoid nodules by IHC.
   Partial remission (PR) requires:
The blood count should show one of the following results:
   Neutrophils more than 1.5×109/L (1500 /μL) without need for exogenous growth factors.
   Platelet counts greater than 100×109/L (100 000 /μL) or 50 percent improvement over baseline without need for exogenous growth factors.
   Hemoglobin greater than 110 g/L (11.0 g/dL) or 50 percent improvement over baseline without requiring red blood cell transfusions or exogenous erythropoietin.
And two of the following three results:
   A decrease in the number of blood lymphocytes by 50 percent or more from the value before therapy.
   Reduction in lymphadenopathy by physical examination or imaging as defined by:
      A decrease in lymph node size by 50 percent or more either in the sum products of up to 6 lymph nodes, or in the largest diameter of the enlarged lymph node(s) detected prior to therapy.
   No increase in any lymph node, and no new enlarged lymph node.
In small lymph nodes (<2 cm), an increase of less than 25 percent is not considered to be significant.
A reduction in splenomegaly and hepatomegaly by 50 percent or more, by physical examination or imaging.

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A method of treating cancer in a patient, comprising administering to the patient an effective amount of (a) a compound of formula IA: 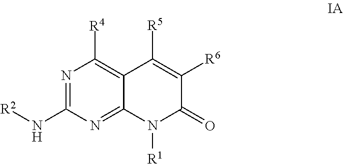
or a metabolite or a pharmaceutically acceptable salt thereof; wherein:
R¹ is alkyl, cycloalkyl, cycloalkylalkyl, aryl, arylalkyl, heterocycloalkyl, heterocycloalkylalkyl, heteroaryl, or heteroarylalkyl;
R² is hydrogen or alkyl;
R⁴ is alkyl;
R⁵ is hydrogen; and
R⁶ is phenyl, acyl, or heteroaryl wherein the phenyl and heteroaryl are is optionally substituted with 1, 2, 3, 4, or 5 R⁹ groups; and
each R⁹, when present, is independently halo, alkyl, haloalkyl, alkoxy, haloalkoxy, cyano, amino, alkylamino, dialkylamino, alkoxyalkyl, carboxyalkyl, alkoxycarbonyl, aminoalkyl, cycloalkyl, aryl, arylalkyl, aryloxy, heterocycloalkyl, or heteroaryl and where the cycloalkyl, aryl, heterocycloalkyl, and heteroaryl, each either alone or as part of another group within R⁹, are independently optionally substituted with 1, 2, 3, or 4 groups selected from halo, alkyl, haloalkyl, hydroxy, alkoxy, haloalkoxy, amino, alkylamino, and dialkylamino,
wherein the cancer is selected from the group consisting of relapsed or refractory NHL, MCL, FL, CLL/SLL, and DLBCL.

2. The method of claim 1, wherein R¹ in the compound of formula IA is alkyl, cycloalkyl, heterocycloalkylalkyl, or arylalkyl; R₂ is hydrogen or alkyl; R⁴ is alkyl; R⁵ is hydrogen; R⁶ is phenyl or heteroaryl wherein the phenyl and heteroaryl are is optionally substituted with one, two, or three R⁹ groups; each R⁸, when present, is independently amino, alkylamino, dialkylamino, or halo; and each R⁸, when present, is independently alkyl, arylalkyl, cyano, aryl, alkoxycarbonyl.

3. The method of claim 1, wherein R⁴ in the compound of formula IA is methyl.

4. The method of claim 1, wherein R¹ in the compound of formula IA is alkyl, cycloalkyl, or heterocycloalkyl.

5. The method of claim 1, wherein R¹ in the compound of formula IA is alkyl.

6. The method of claim 5, wherein R⁶ in the compound of formula IA is heteroaryl optionally substituted with 1, 2, or 3 R⁹ groups.

7. The method of claim 6, wherein R⁶ in the compound of formula IA is pyrazolyl, imidazolyl, thienyl, thiazolyl, oxazolyl, isoxazolyl, oxadiazolyl, furanyl, pyrrolyl, triazolyl, or tetrazolyl; each of which is optionally substituted with 1, 2, or 3 R⁹ groups.

8. The method of claim 7, wherein R⁶ in the compound of formula IA is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with 1, 2, or 3 R⁹ groups.

9. The method of claim 1, wherein R² in the compound of formula IA is hydrogen, R⁴ is methyl, R¹ is optionally substituted alkyl, cycloalkyl, or heterocycloalkyl, and R⁶ is heteroaryl optionally substituted with 1, 2, or 3 R⁹ groups.

10. The method of claim 1 wherein the compound of formula IA is selected from:
2-amino-8-ethyl-4-methyl-6-(1H-pyrazol-5-yl)pyrido[2,3-d]pyrimidin-7(8H)-one;
2-amino-8-cyclopentyl-4-methyl-6-(1H-pyrazol-3-yl)pyrido[2,3-d]pyrimidin-7(8H)-one;
2-amino-4-methyl-8-(1-methylethyl)-6-(1H-pyrazol-3-yl)pyrido[2,3-d]pyrimidin-7(8H)-one;
2-amino-4-methyl-8-(phenylmethyl)-6-(1H-pyrazol-3-yl)pyrido[2,3-d]pyrimidin-7(8H)-one;
2-amino-8-ethyl-4-methyl-6-(4-methyl-3-thienyl)pyrido[2,3-d]pyrimidin-7(8H)-one;
2-amino-8-ethyl-4-methyl-6-(2-thienyl)pyrido[2,3-d]pyrimidin-7(8H)-one;
2-amino-8-ethyl-4-methyl-6-(3-thienyl)pyrido[2,3-d]pyrimidin-7(8H)-one;
2-amino-8-ethyl-6-furan-3-yl-4-methylpyrido[2,3-d]pyrimidin-7(8H)-one;
2-amino-8-ethyl-4-methyl-6-phenylpyrido[2,3-d]pyrimidin-7(8H)-one;
2-amino-8-ethyl-6-isoxazol-4-yl-4-methylpyrido[2,3-d]pyrimidin-7(8H)-one;
2-amino-8-ethyl-6-furan-2-yl-4-methylpyrido[2,3-d]pyrimidin-7(8H)-one;
5-(2-amino-8-ethyl-4-methyl-7-oxo-7,8-dihydropyrido[2,3-d]pyrimidin-6-yl)thiophene-2-carbonitrile;
2-amino-8-ethyl-4-methyl-6-pyrimidin-5-ylpyrido[2,3-d]pyrimidin-7(8H)-one;
2-amino-8-ethyl-6-(1H-imidazol-5-yl)-4-methylpyrido[2,3-d]pyrimidin-7(8H)-one;
2-amino-8-ethyl-4-methyl-6-(1H-1,2,3-triazol-5-yl)pyrido[2,3-d]pyrimidin-7(8H)-one;
2-amino-8-ethyl-4-methyl-6-(1H-pyrazol-4-yl)pyrido[2,3-d]pyrimidin-7(8H)-one;
2-amino-8-ethyl-4-methyl-6-(1,3-thiazol-2-yl)pyrido[2,3-d]pyrimidin-7(8H)-one;
2-amino-8-ethyl-4-methyl-6-(1H-tetrazol-5-yl)pyrido[2,3-d]pyrimidin-7(8H)-one;
2-amino-8-ethyl-4-methyl-6-(1-methyl-1H-pyrrol-2-yl)pyrido[2,3-d]pyrimidin-7(8H)-one;
2-amino-4,8-diethyl-6-(1H-pyrazol-5-yl)pyrido[2,3-d]pyrimidin-7(8H)-one; and
2-amino-8-cyclopentyl-4-methyl-6-(1,3-thiazol-5-yl)pyrido[2,3-d]pyrimidin-7(8H)-one.

11. The method of claim 10, wherein the compound of formula IA is 2-amino-8-ethyl-4-methyl-6-(1H-pyrazol-5-yl)pyrido[2,3-d]pyrimidin-7(8H)-one or a pharmaceutically acceptable salt thereof.

12. A method of treating a lymphoproliferative malignancy in a human patient, comprising administering to the patient an effective amount of 2-amino-8-ethyl-4-methyl-6-(1H-pyrazol-5-yl)pyrido[2,3-d]pyrimidin-7(8H)-one or a pharmaceutically acceptable salt thereof; wherein the method comprises at least one dosing cycle, wherein the dosing cycle is a period of 28 days.

13. The method of claim 12, wherein 2-amino-8-ethyl-4-methyl-6-(1H-pyrazol-5-yl)pyrido[2,3-d]pyrimidin-7(8H)-one or pharmaceutically acceptable salt thereof is administered at about 50 mg BID.

14. The method of claim 13, wherein the lymphoproliferative malignancy is selected from the group consisting of relapsed or refractory NHL, MCL, FL, CLL/SLL, and DLBCL.

15. The method of claim 1, wherein the effective amount produces at least one therapeutic effect selected from the group consisting of reduction in size of a tumor, reduction in metastasis, complete remission, partial remission, stable disease, increase in overall response rate, or a pathologic complete response.

16. The method of claim 1, wherein the effective amount produces an improved clinical benefit rate (CBR) according to the equation CBR=CR (complete remission)+PR (partial remission)+SD (stable disease)≧6 months) as compared to other treatments.

17. The method of claim 15, wherein the improvement of clinical benefit rate is about 20 percent or higher.

18. The method of claim 17, the therapeutic effect is an increase in overall response rate.

19. The method of claim 18, wherein the increase in overall response rate is about 10 percent or more.

20. The method of claim 19, wherein a comparable clinical benefit rate (CBR) according to the equation CBR=CR (complete remission)+PR (partial remission)+SD (stable disease)≧6 dosing cycles) is obtained with treatment of a) Compound A or a pharmaceutically acceptable salt thereof, as compared to other treatments administered without Compound A.

21. The method of claim 19, wherein the improvement of clinical benefit rate is at least about 20 percent.

22. The method of claim 21, wherein a comparable clinical benefit rate (CBR=CR (complete remission)+PR (partial remission)+SD (stable disease)≧6 months) is obtained with treatment of a) Compound A or a pharmaceutically acceptable salt thereof as compared to other treatments without Compound A.

23. The method of claim 22, wherein the improvement of clinical benefit rate is at least about 20 percent.

24. A method of treating a lymphoproliferative malignancy in a human patient, comprising administering a composition comprising an effective amount of 2-amino-8-ethyl-4-methyl-6-(1H-pyrazol-5-yl)pyrido[2,3-d]pyrimidin-7(8H)-one or a pharmaceutically acceptable salt thereof.

25. The composition of claim 24, wherein the 2-amino-8-ethyl-4-methyl-6-(1H-pyrazol-5-yl)pyrido[2,3-d]pyrimidin-7(8H)-one or a pharmaceutically acceptable salt thereof is formulated for a dose of about 50 mg BID.

26. The composition of claim 24, wherein the lymphoproliferative malignancy is selected from the group consisting of relapsed or refractory NHL, MCL, FL, CLL/SLL and DLBCL.
